(19) United States
(12) Patent Application Publication
(10) Pub. No.: US 2012/0251575 A1
(43) Pub. Date: Oct. 4, 2012

(54) Endopeptidase Treatment of Involuntary Movement Disorders

(75) Inventors: Andrew M. Blumenfeld, Del Mar, CA (US); Mitchell F. Brin, Newport Beach, CA (US)

(73) Assignee: ALLERGAN, INC., Irvine, CA (US)

(21) Appl. No.: 13/425,253

(22) Filed: Mar. 20, 2012

Related U.S. Application Data
(60) Provisional application No. 61/468,332, filed on Mar. 28, 2011.

(51) Int. Cl. A61K 39/08; C12N 9/96; A61P 25/14

(52) U.S. Cl. 424/239.1; 435/188

(57) ABSTRACT

The present specification discloses TEMs, compositions comprising such TEMs, compositions comprising such TEMs and Clostridial toxins, methods of treating an involuntary movement disorder in an individual using such compositions, use of such TEMs in manufacturing a medicament for treating an involuntary movement disorder, use of such TEMs and Clostridial toxins in manufacturing a medicament for treating an involuntary movement disorder, use of such TEMs in treating an involuntary movement disorder, and use of such TEMs and Clostridial toxins in treating an involuntary movement disorder.

This application claims the benefit of priority pursuant to 35 U.S.C. §119(e) to U.S. provisional patent application Ser. No. 61/468,332, filed Mar. 28, 2011, incorporated entirely by reference.

Involuntary movement disorders are a group of conditions with varied causes and overlapping symptoms. To date, treatments with broad applicability in this area have not been developed.

The ability of Clostridial toxins, such as, e.g., Botulinum neurotoxins (BoNTs), BoNT/A, BoNT/B, BoNT/C1, BoNT/D, BoNT/E, BoNT/F and BoNT/G, and Tetanus neurotoxin (TeNT), to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). Clostridial toxins commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea), BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Solstice Neurosciences, Inc., South San Francisco, Calif.). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder.

Clostridial toxin therapies have been successfully used to treat involuntary muscle disorders. However, toxin administration in some applications can be challenging because of the larger doses required to achieve a beneficial effect. Larger doses can increase the likelihood that the toxin may move through the interstitial fluids and the circulatory systems, such as, e.g., the cardiovascular system and the lymphatic system, of the body, resulting in the undesirable dispersal of the toxin to areas not targeted for toxin treatment. Such dispersal can lead to undesirable side effects, such as, e.g., inhibition of neurotransmitter release in neurons not targeted for treatment or paralysis of a muscle not targeted for treatment. For example, an individual administered a therapeutically effective amount of a BoNT/A treatment into the neck muscles for cervical dystonia may develop dysphagia because of dispersal of the toxin into the oropharynx. Thus, there still remains a need for involuntary movement disorder treatments having the therapeutic effects that only larger doses of a Clostridial toxin can currently provide, but reduce or prevent the undesirable side-effects associated with larger doses of a Clostridial toxin administration.

A Clostridial toxin treatment inhibits neurotransmitter release by disrupting the exocytotic process used to secret the neurotransmitter into the synaptic cleft. There is a great desire by the pharmaceutical industry to expand the use of Clostridial toxin therapies beyond its current myo-relaxant applications to treat sensory, sympathetic, and/or parasympathetic nerve-based ailments, such as, e.g., various kinds of involuntary movement disorders. One approach that is currently being exploited involves modifying a Clostridial toxin such that the modified toxin has an altered cell targeting capability for a neuronal or non-neuronal cell of interest. Called re-targeted endopeptidases or Targeted Vesicular Exocytosis Modulator Proteins (TVEMPs) or Targeted Exocytosis Modulators (TEMs), these molecules achieve their exocytosis inhibitory effects by targeting a receptor present on the neuronal or non-neuronal target cell of interest. This re-targeted capability is achieved by replacing the naturally-occurring binding domain of a Clostridial toxin with a targeting domain showing a selective binding activity for a non-Clostridial toxin receptor present in a cell of interest. Such modifications to the binding domain result in a molecule that is able to selectively bind to a non-Clostridial toxin receptor present on the target cell. A re-targeted endopeptidase can bind to a target receptor, translocate into the cytoplasm, and exert its proteolytic effect on the SNARE complex of the neuronal or non-neuronal target cell of interest.

The present specification discloses TEMs, compositions comprising TEMs, and methods for treating an individual suffering from an involuntary movement disorder. This is accomplished by administering a therapeutically effective amount of a composition comprising a TEM to an individual in need thereof. The disclosed methods provide a safe, inexpensive, out patient-based treatment for the treatment of involuntary movement disorders. In addition, the therapies disclosed herein reduce or prevent unwanted side-effects associated with larger Clostridial toxin doses. These and related advantages are useful for various clinical applications, such as, e.g., the treatment of involuntary movement disorders where a larger amount of a Clostridial toxin to an individual could produce a beneficial effect, but for the undesirable side-effects.

### SUMMARY

With reference to involuntary movement disorders as disclosed herein, and without wishing to be limited by any particular theory, it is believed that sympathetic, parasympathetic, and/or sensory neurons have important functions in aspects of involuntary muscle function and that improper innervations from these types of neurons can contribute to one or more different types of involuntary movement disorders. As such, TEMs comprising a targeting domain for a receptor present on sympathetic, parasympathetic, and/or sensory neurons can reduce or prevent these improper innervations, thereby reducing or preventing one or more symptoms associate with an involuntary movement disorder. It is further theorized that such a TEM in combination with a Clostridial toxin can provide enhanced, if not synergistic, therapeutic benefit because such a combination also inhibit motor neurons. However, using a combination therapy of such a TEM with a Clostridial toxin, also allows a lower dose of a Clostridial toxin to be administered to treat an involuntary movement disorder. This will result in a decrease in muscle weakness generated in the compensatory muscles relative to the current treatment paradigm. As such, a combined therapy using a Clostridial toxin and a TEM comprising a targeting domain for a receptor present on sympathetic, parasympathetic, and/or sensory neurons can reduce or prevent these improper innervations, and in combination can reduce or prevent one or more symptoms associate with an involuntary movement disorder.

Thus, aspects of the present specification disclose methods of treating an involuntary movement disorder in an individual, the methods comprising the step of administering to the individual in need thereof a therapeutically effective amount of a composition including a TEM, wherein administration of the composition reduces a symptom of the involuntary movement disorder, thereby treating the individual. In some aspects, a TEM may comprise a targeting domain, a Clostridial toxin translocation domain and a Clostridial toxin enzymatic domain. In some aspects, a TEM may comprise a targeting domain, a Clostridial toxin translocation domain, a Clostridial toxin enzymatic domain, and an exogenous protease cleavage site. A targeting domain includes, without limitation, a sensory neuron targeting domain, a sympathetic neuron targeting domain, or a parasympathetic neuron targeting domain. An involuntary movement disorder includes, without limitation, an ataxia, a chorea, a dyskinesia, a synkinesia, a dystonia, a cerebral palsy, a periodic limb movement disorder, a restless leg syndrome, a spasm, a stereotypy, a tic, and a tremor.

Other aspects of the present specification disclose uses of a TEM disclosed herein in the manufacturing a medicament for treating an involuntary movement disorder disclosed herein in an individual in need thereof.

Yet other aspects of the present specification uses of a TEM disclosed herein in the treatment of an involuntary movement disorder disclosed herein in an individual in need thereof.

Other aspects of the present specification disclose methods of treating an involuntary movement disorder in an individual, the methods comprising the step of administering to the individual in need thereof a therapeutically effective amount of a composition including a Clostridial neurotoxin and a TEM, wherein administration of the composition reduces a symptom of the involuntary movement, thereby treating the individual. A Clostridial neurotoxin includes, without limitation, a Botulinum toxin (BoNT), a Tetanus toxin (TeNT), a Baratii toxin (BaNT), and a Butyricum toxin (BuNT). In some aspects, a TEM may comprise a targeting domain, a Clostridial toxin translocation domain and a Clostridial toxin enzymatic domain. In some aspects, a TEM may comprise a targeting domain, a Clostridial toxin translocation domain, a Clostridial toxin enzymatic domain, and an exogenous protease cleavage site. A targeting domain includes, without limitation, a sensory neuron targeting domain, a sympathetic neuron targeting domain, or a parasympathetic neuron targeting domain. An involuntary movement disorder includes, without limitation, an ataxia, a chorea, a dyskinesia, a synkinesia, a dystonia, a cerebral palsy, a periodic limb movement disorder, a restless leg syndrome, a spasm, a stereotypy, a tic, and a tremor.

Other aspects of the present specification disclose uses of a Clostridial neurotoxin and a TEM disclosed herein in the manufacturing a medicament for treating an involuntary movement disorder disclosed herein in an individual in need thereof.

Yet other aspects of the present specification uses of a Clostridial neurotoxin and a TEM disclosed herein in the treatment of an involuntary movement disorder disclosed herein in an individual in need thereof.

### BRIEF DESCRIPTION OF THE DRAWINGS

FIG. 1 shows a schematic of the current paradigm of neurotransmitter release and Clostridial toxin intoxication in a central and peripheral neuron. FIG. 1A shows a schematic for the neurotransmitter release mechanism of a central and peripheral neuron. The release process can be described as comprising two steps: 1) vesicle docking, where the vesicle-bound SNARE protein of a vesicle containing neurotransmitter molecules associates with the membrane-bound SNARE proteins located at the plasma membrane; and 2) neurotransmitter release, where the vesicle fuses with the plasma membrane and the neurotransmitter molecules are exocytosed. FIG. 1B shows a schematic of the intoxication mechanism for tetanus and botulinum toxin activity in a central and peripheral neuron. This intoxication process can be described as comprising four steps: 1) receptor binding, where a Clostridial toxin binds to a Clostridial receptor system and initiates the intoxication process; 2) complex internalization, where after toxin binding, a vesicle containing the toxin/receptor system complex is endocytosed into the cell; 3) light chain translocation, where multiple events are thought to occur, including, e.g., changes in the internal pH of the vesicle, formation of a channel pore comprising the HN domain of the Clostridial toxin heavy chain, separation of the Clostridial toxin light chain from the heavy chain, and release of the active light chain and 4) enzymatic target modification, where the activate light chain of Clostridial toxin proteolytically cleaves its target SNARE substrate, such as, e.g., SNAP-25, VAMP or Syntaxin, thereby preventing vesicle docking and neurotransmitter release.

FIG. 2 shows the domain organization of naturally-occurring Clostridial toxins. The single-chain form depicts the amino to carboxyl linear organization comprising an enzymatic domain, a translocation domain, and a retargeted peptide binding domain. The di-chain loop region located between the translocation and enzymatic domains is depicted by the double SS bracket. This region comprises an endogenous di-chain loop protease cleavage site that upon proteolytic cleavage with a naturally-occurring protease, such as, e.g., an endogenous Clostridial toxin protease or a naturally-occurring protease produced in the environment, converts the single-chain form of the toxin into the di-chain form. Above the single-chain form, the H_{CC} region of the Clostridial toxin binding domain is depicted. This region comprises the β-trefoil domain which comprises in an amino to carboxyl linear organization an α-fold, a β4/β5 hairpin turn, a β-fold, a β8/β9 hairpin turn and a γ-fold.

FIG. 3 shows TEM domain organization with a targeting domain located at the amino terminus of a TEM. FIG. 3A depicts the single-chain polypeptide form of a TEM with an amino to carboxyl linear organization comprising a targeting domain, a translocation domain, a di-chain loop region comprising an exogenous protease cleavage site (P), and an enzymatic domain. Upon proteolytic cleavage with a P protease, the single-chain form of the TEM is converted to the di-chain form. FIG. 3B depicts the single polypeptide form of a TEM with an amino to carboxyl linear organization comprising a targeting domain, an enzymatic domain, a di-chain loop region comprising an exogenous protease cleavage site (P), and a translocation domain. Upon proteolytic cleavage with a P protease, the single-chain form of the TEM is converted to the di-chain form.

FIG. 4 shows a TEM domain organization with a targeting domain located between the other two domains. FIG. 4A depicts the single polypeptide form of a TEM with an amino to carboxyl linear organization comprising an enzymatic domain, a di-chain loop region comprising an exogenous protease cleavage site (P), a targeting domain, and a translocation domain. Upon proteolytic cleavage with a P protease, the single-chain form of the TEM is converted to the di-chain form. FIG. 4B depicts the single polypeptide form of a TEM with an amino to carboxyl linear organization comprising a translocation domain, a di-chain loop region comprising an exogenous protease cleavage site (P), a targeting domain, and an enzymatic domain. Upon proteolytic cleavage with a P protease, the single-chain form of the TEM is converted to the di-chain form. FIG. 4C depicts the single polypeptide form of a TEM with an amino to carboxyl linear organization comprising an enzymatic domain, a targeting domain, a di-chain loop region comprising an exogenous protease cleavage site (P), and a translocation domain. Upon proteolytic cleavage with a P protease, the single-chain form of the TEM is converted to the di-chain form. FIG. 4D depicts the single polypeptide form of a TEM with an amino to carboxyl linear organization comprising a translocation domain, a targeting domain, a di-chain loop region comprising an exogenous protease cleavage site (P), and an enzymatic domain. Upon proteolytic cleavage with a P protease, the single-chain form of the TEM is converted to the di-chain form.

FIG. 5 shows a TEM domain organization with a targeting domain located at the carboxyl terminus of the TEM. FIG. 5A depicts the single polypeptide form of a TEM with an amino to carboxyl linear organization comprising an enzymatic domain, a di-chain loop region comprising an exogenous protease cleavage site (P), a translocation domain, and a targeting domain. Upon proteolytic cleavage with a P protease, the single-chain form of the TEM is converted to the di-chain form. FIG. 5B depicts the single polypeptide form of a TEM with an amino to carboxyl linear organization comprising a translocation domain, a di-chain loop region comprising an exogenous protease cleavage site (P), an enzymatic domain, and a targeting domain. Upon proteolytic cleavage with a P protease, the single-chain form of the TEM is converted to the di-chain form.

### DESCRIPTION

Clostridia toxins produced by Clostridium botulinum, Clostridium tetani, Clostridium baratii and Clostridium butyricum are the most widely used in therapeutic and cosmetic treatments of humans and other mammals. Strains of C. botulinum produce seven antigenically-distinct types of Botulinum toxins (BoNTs), which have been identified by investigating botulism outbreaks in man (BoNT/A, BoNT/B, BoNT/E and BoNT/F), animals (BoNT/C1 and BoNT/D), or isolated from soil (BoNT/G). BoNTs possess approximately 35% amino acid identity with each other and share the same functional domain organization and overall structural architecture. It is recognized by those of skill in the art that within each type of Clostridial toxin there can be subtypes that differ somewhat in their amino acid sequence, and also in the nucleic acids encoding these proteins. For example, there are presently five BoNT/A subtypes, BoNT/A1, BoNT/A2, BoNT/A3 BoNT/A4 and BoNT/A5, with specific subtypes showing approximately 89% amino acid identity when compared to another BoNT/A subtype. While all seven BoNT serotypes have similar structure and pharmacological properties, each also displays heterogeneous bacteriological characteristics. In contrast, tetanus toxin (TeNT) is produced by a uniform group of C. tetani. Two other Clostridia species, C. baratii and C. butyricum, produce toxins, BaNT and BuNT, which are functionally similar to BoNT/F and BoNT/E, respectively.

Clostridial toxins are released by Clostridial bacterium as complexes comprising the approximately 150-kDa Clostridial toxin along with associated non-toxin proteins (NAPs). Identified NAPs include proteins possessing hemagglutination activity, such, e.g., a hemagglutinin of approximately 17-kDa (HA-17), a hemagglutinin of approximately 33-kDa (HA-33) and a hemagglutinin of approximately 70-kDa (HA-70); as well as non-toxic non-hemagglutinin (NTNH), a protein of approximately 130-kDa. Thus, the botulinum toxin type A complex can be produced by Clostridial bacterium as 900-kDa, 500-kDa and 300-kDa forms. Botulinum toxin types B and C₁ are apparently produced as only a 500-kDa complex. Botulinum toxin type D is produced as both 300-kDa and 500-kDa complexes. Finally, botulinum toxin types E and F are produced as only approximately 300-kDa complexes. The differences in molecular weight for the complexes are due to differing ratios of NAPs. The toxin complex is important for the intoxication process because it provides protection from adverse environmental conditions, resistance to protease digestion, and appears to facilitate internalization and activation of the toxin.

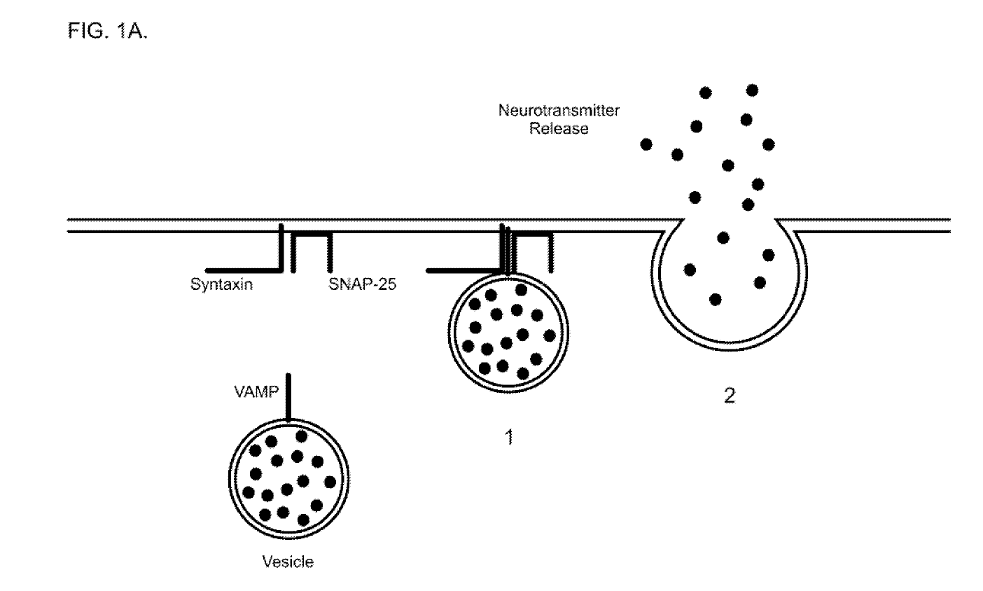

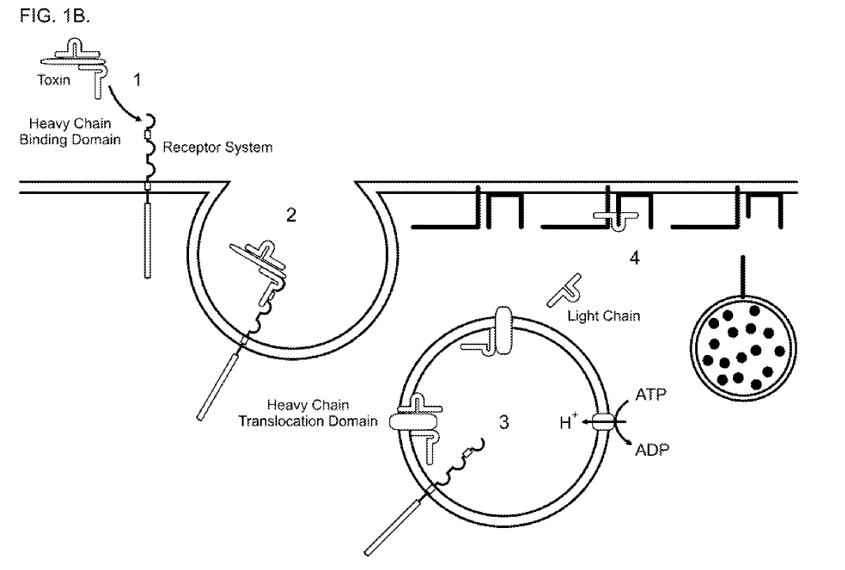

A Clostridial toxin itself is translated as a single chain polypeptide that is subsequently cleaved by proteolytic scission within a disulfide loop by a naturally-occurring protease (FIG. 1). This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains. The naturally-occurring protease used to convert the single chain molecule into the di-chain is currently not known. In some serotypes, such as, e.g., BoNT/A, the naturally-occurring protease is produced endogenously by the bacteria serotype and cleavage occurs within the cell before the toxin is release into the environment. However, in other serotypes, such as, e.g., BoNT/E, the bacterial strain appears not to produce an endogenous protease capable of converting the single chain form of the toxin into the di-chain form. In these situations, the toxin is released from the cell as a single-chain toxin which is subsequently converted into the di-chain form by a naturally-occurring protease found in the environment.

Each mature di-chain molecule of a Clostridial toxin comprises three functionally distinct domains: 1) an enzymatic domain located in the light chain (LC) that includes a metalloprotease region containing a zinc-dependent endopeptidase activity which specifically targets core components of the neurotransmitter release apparatus; 2) a translocation domain contained within the amino-terminal half of the heavy chain (H_{N}) that facilitates release of the LC from intracellular vesicles into the cytoplasm of the target cell; and 3) a binding domain found within the carboxyl-terminal half of the heavy chain (HO that determines the binding activity and binding specificity of the toxin to the receptor complex located at the surface of the target cell. The H_{C} domain comprises two distinct structural features of roughly equal size that indicate function and are designated the H_{CN} and H_{CC} subdomains.

Clostridial toxins act on the nervous system by blocking the release of acetylcholine (ACh) at the pre-synaptic neuromuscular junction. The binding, translocation and enzymatic activity of these three functional domains are all necessary for toxicity. While all details of this process are not yet precisely known, the overall cellular intoxication mechanism whereby Clostridial toxins enter a neuron and inhibit neurotransmitter release is similar, regardless of serotype or subtype. Although applicants have no wish to be limited by the following description, the intoxication mechanism can be described as comprising at least four steps: 1) receptor binding, 2) complex internalization, 3) light chain translocation, and 4) enzymatic target modification (FIG. 1). The process is initiated when the binding domain of a Clostridial toxin binds to a toxin-specific receptor system located on the plasma membrane surface of a target cell. The binding specificity of a receptor complex is thought to be achieved, in part, by specific combinations of gangliosides and protein receptors that appear to distinctly comprise each Clostridial toxin receptor complex. Once bound, the toxin/receptor complexes are internalized by endocytosis and the internalized vesicles are sorted to specific intracellular routes. The translocation step appears to be triggered by the acidification of the vesicle compartment. This process seems to initiate pH-dependent structural rearrangements that increase hydrophobicity, create a pore in the vesicle membrane, and promote formation of the di-chain form of the toxin. Once di-chain formation occurs, light chain endopeptidase of the toxin is released from the intracellular vesicle via the pore into the cytosol where it appears to specifically target one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A and BoNT/E cleave SNAP-25 in the carboxyl-terminal region, releasing a nine or twenty-six amino acid segment, respectively, and BoNT/C1 also cleaves SNAP-25 near the carboxyl-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface.

Aspects of the present specification disclose, in part, in part, a Clostridial toxin. As used herein, the term “Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/C₁, a BoNT/D, a BoNT/E, a BoNT/F, a BoNT/G, a Tetanus toxin (TeNT), a Baratii toxin (BaNT), and a Butyricum toxin (BuNT). The BoNT/C₂ cytotoxin and BoNT/C₃ cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof.

A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

Clostridial toxins can be produced using standard purification or recombinant biology techniques known to those skilled in the art. See, e.g., Hui Xiang et al., Animal Product Free System and Process for Purifying a Botulinum Toxin, U.S. Pat. No. 7,354,740, which is hereby incorporated by reference in its entirety. For example, a BoNT/A complex can be isolated and purified from an anaerobic fermentation by cultivating Clostridium botulinum type A in a suitable medium. Raw toxin can be harvested by precipitation with sulfuric acid and concentrated by ultramicrofiltration. Purification can be carried out by dissolving the acid precipitate in calcium chloride. The toxin can then be precipitated with cold ethanol. The precipitate can be dissolved in sodium phosphate buffer and centrifuged. Upon drying there can then be obtained approximately 900 kD crystalline BoNT/A complex with a specific potency of 3×10⁷ LD₅₀ U/mg or greater. Furthermore, NAPs can be separated out to obtain purified toxin, such as e.g., BoNT/A with an approximately 150 kD molecular weight with a specific potency of 1−2×10⁸ LD₅₀ U/mg or greater, purified BoNT/B with an approximately 156 kD molecular weight with a specific potency of 1−2×10⁸ LD₅₀ U/mg or greater, and purified BoNT/F with an approximately 155 kD molecular weight with a specific potency of 1−2×10⁷ LD₅₀ U/mg or greater. See Edward J. Schantz & Eric A. Johnson, Properties and use of Botulinum Toxin and Other Microbial Neurotoxins in Medicine, Microbiol Rev. 56: 80-99 (1992), which is hereby incorporated in its entirety. As another example, recombinant Clostridial toxins can be recombinantly produced as described in Steward et al., Optimizing Expression of Active Botulinum Toxin Type A, U.S. Patent Publication 2008/0057575; and Steward et al., Optimizing Expression of Active Botulinum Toxin Type E, U.S. Patent Publication 2008/0138893, each of which is hereby incorporated in its entirety.

Clostridial toxins are also commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea), BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Solstice Neurosciences, Inc., South San Francisco, Calif.). Clostridial toxin complexes may be obtained from, e.g., List Biological Laboratories, Inc. (Campbell, Calif.), the Centre for Applied Microbiology and Research (Porton Down, U.K), Wako (Osaka, Japan), and Sigma Chemicals (St Louis, Mo.).

In an embodiment, a Clostridial may be a Botulinum toxin, Tetanus toxin, a Baratii toxin, or a Butyricum toxin. In aspects of this embodiment, a Botulinum toxin may be a BoNT/A, a BoNT/B, a BoNT/C₁, a BoNT/D, a BoNT/E, a BoNT/F, or a BoNT/G. In another embodiment, a Clostridial toxin may be a Clostridial toxin variant. In aspects of this embodiment, a Clostridial toxin variant may be a naturally-occurring Clostridial toxin variant or a non-naturally-occurring Clostridial toxin variant. In other aspects of this embodiment, a Clostridial toxin variant may be a BoNT/A variant, a BoNT/B variant, a BoNT/C₁ variant, a BoNT/D variant, a BoNT/E variant, a BoNT/F variant, a BoNT/G variant, a TeNT variant, a BaNT variant, or a BuNT variant, where the variant is either a naturally-occurring variant or a non-naturally-occurring variant.

In an embodiment, a Clostridial toxin may be a Clostridial toxin complex. In aspects of this embodiment, a Clostridial toxin complex may be a BoNT/A complex, a BoNT/B complex, a BoNT/C₁ complex, a BoNT/D complex, a BoNT/E complex, a BoNT/F complex, a BoNT/G complex, a TeNT complex, a BaNT complex, or a BuNT complex. In other aspects of this embodiment, a Clostridial toxin complex may be a 900-kDa BoNT/A complex, a 500-kDa BoNT/A complex, a 300-kDa BoNT/A complex, a 500-kDa BoNT/B complex, a 500-kDa BoNT/C1 complex, a 500-kDa BoNT/D complex, a 300-kDa BoNT/D complex, a 300-kDa BoNT/E complex, or a 300-kDa BoNT/F complex.

Aspects of the present disclosure comprise, in part, a Targeted Exocytosis Modulator. As used herein, the term “Targeted Exocytosis Modulator” is synonymous with “TEM” or “retargeted endopeptidase.” Generally, a TEM comprises an enzymatic domain from a Clostridial toxin light chain, a translocation domain from a Clostridial toxin heavy chain, and a targeting domain. The targeting domain of a TEM provides an altered cell targeting capability that targets the molecule to a receptor other than the native Clostridial toxin receptor utilized by a naturally-occurring Clostridial toxin. This re-targeted capability is achieved by replacing the naturally-occurring binding domain of a Clostridial toxin with a targeting domain having a binding activity for a non-Clostridial toxin receptor. Although binding to a non-Clostridial toxin receptor, a TEM undergoes all the other steps of the intoxication process including internalization of the TEM/receptor complex into the cytoplasm, formation of the pore in the vesicle membrane and di-chain molecule, translocation of the enzymatic domain into the cytoplasm, and exerting a proteolytic effect on a component of the SNARE complex of the target cell.

However, an important difference between TEMs, such as, e.g., TEMs disclosed herein, and native Clostridial toxins is that since TEMs do not target motor neurons, the lethality associated with over-dosing an individual with a TEM is greatly minimized, if not avoided altogether. For example, a TEM comprising an opioid targeting domain can be administered at 10,000 times the therapeutically effective dose before evidence of lethality is observed, and this lethality is due to the passive diffusion of the molecule and not via the intoxication process. Thus, for all practical purposes TEMs are non-lethal molecules.

As used herein, the term “Clostridial toxin enzymatic domain” refers to a Clostridial toxin polypeptide located in the light chain of a Clostridial toxin that executes the enzymatic target modification step of the intoxication process. A Clostridial toxin enzymatic domain includes a metalloprotease region containing a zinc-dependent endopeptidase activity which specifically targets core components of the neurotransmitter release apparatus. Thus, a Clostridial toxin enzymatic domain specifically targets and proteolytically cleavages of a Clostridial toxin substrate, such as, e.g., SNARE proteins like a SNAP-25 substrate, a VAMP substrate and a Syntaxin substrate.

A Clostridial toxin enzymatic domain includes, without limitation, naturally occurring Clostridial toxin enzymatic domain variants, such as, e.g., Clostridial toxin enzymatic domain isoforms and Clostridial toxin enzymatic domain subtypes; non-naturally occurring Clostridial toxin enzymatic domain variants, such as, e.g., conservative Clostridial toxin enzymatic domain variants, non-conservative Clostridial toxin enzymatic domain variants, Clostridial toxin enzymatic domain chimeras, active Clostridial toxin enzymatic domain fragments thereof, or any combination thereof. Non-limiting examples of a Clostridial toxin enzymatic domain include, e.g., a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, and a BuNT enzymatic domain.

As used herein, the term “Clostridial toxin translocation domain” refers to a Clostridial toxin polypeptide located within the amino-terminal half of the heavy chain of a Clostridial toxin that executes the translocation step of the intoxication process. The translocation step appears to involve an allosteric conformational change of the translocation domain caused by a decrease in pH within the intracellular vesicle. This conformational change results in the formation of a pore in the vesicular membrane that permits the movement of the light chain from within the vesicle into the cytoplasm. Thus, a Clostridial toxin translocation domain facilitates the movement of a Clostridial toxin light chain across a membrane of an intracellular vesicle into the cytoplasm of a cell.

A Clostridial toxin translocation domain includes, without limitation, naturally occurring Clostridial toxin translocation domain variants, such as, e.g., Clostridial toxin translocation domain isoforms and Clostridial toxin translocation domain subtypes; non-naturally occurring Clostridial toxin translocation domain variants, such as, e.g., conservative Clostridial toxin translocation domain variants, non-conservative Clostridial toxin translocation domain variants, Clostridial toxin translocation domain chimerics, active Clostridial toxin translocation domain fragments thereof, or any combination thereof. Non-limiting examples of a Clostridial toxin translocation domain include, e.g., a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, and a BuNT translocation domain.

As used herein, the term “targeting domain” is synonymous with “binding domain” or “targeting moiety” and refers to a polypeptide that executes the receptor binding and/or complex internalization steps of the intoxication process, with the proviso that the binding domain is not a Clostridial toxin binding domain found within the carboxyl-terminal half of the heavy chain of a Clostridial toxin. A targeting domain includes a receptor binding region that confers the binding activity and/or specificity of the targeting domain for its cognate receptor. As used herein, the term “cognate receptor” refers to a receptor for which the targeting domain preferentially interacts with under physiological conditions, or under in vitro conditions substantially approximating physiological conditions. As used herein, the term “preferentially interacts” is synonymous with “preferentially binding” and refers to an interaction that is statistically significantly greater in degree relative to a control. With reference to a targeting domain disclosed herein, a targeting domain binds to its cognate receptor to a statistically significantly greater degree relative to a non-cognate receptor. Said another way, there is a discriminatory binding of the targeting domain to its cognate receptor relative to a non-cognate receptor. Thus, a targeting domain directs binding to a TEM-specific receptor located on the plasma membrane surface of a target cell.

In an embodiment, a targeting domain disclosed herein has an association rate constant that confers preferential binding to its cognate receptor. In aspects of this embodiment, a targeting domain disclosed herein binds to its cognate receptor with an association rate constant of, e.g., less than 1×10⁵ M⁻¹ s⁻¹, less than 1×10⁶ M⁻¹ s⁻¹, less than 1×10⁷ M⁻¹ s⁻¹, or less than 1×10⁸ M⁻¹ s⁻¹. In other aspects of this embodiment, a targeting domain disclosed herein binds to its cognate receptor with an association rate constant of, e.g., more than 1×10⁵ M⁻¹ s⁻¹, more than 1×10⁶ M⁻¹ s⁻¹, more than 1×10⁷ M¹, or more than 1×10⁸ M⁻¹ s⁻¹. In yet other aspects of this embodiment, a targeting domain disclosed herein binds to its cognate receptor with an association rate constant between 1×10⁵ M⁻¹ s⁻¹ to 1×10⁸ M⁻¹ 1×10⁶ M⁻¹ s⁻¹ to 1×10⁸ M⁻¹×10⁵ M⁻¹ s⁻¹ to 1×10⁷ M⁻¹ s⁻¹, or 1×10⁶ M⁻¹ s⁻¹ to 1×10⁷ M⁻¹ s⁻¹.

In another embodiment, a targeting domain disclosed herein has an association rate constant that is greater for its cognate target receptor relative to a non-cognate receptor. In other aspects of this embodiment, a targeting domain disclosed herein has an association rate constant that is greater for its cognate target receptor relative to a non-cognate receptor by, at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has an association rate constant that is greater for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10,000-fold, or about 10-fold to about 100,000-fold.

In yet another embodiment, a targeting domain disclosed herein has a disassociation rate constant that confers preferential binding to its cognate receptor. In other aspects of this embodiment, a targeting domain disclosed herein binds to its cognate receptor with a disassociation rate constant of less than 1×10⁻³ s⁻¹, less than 1×10⁻⁴ s⁻¹, or less than 1×10⁻⁵ s⁻¹. In yet other aspects of this embodiment, a targeting domain disclosed herein binds to its cognate receptor with a disassociation rate constant of, e.g., less than 1.0×10⁻⁴ s⁻¹, less than 2.0×10⁻⁴ s⁻¹, less than 3.0×10⁻⁴ s⁻¹, less than 4.0×10⁻⁴ s⁻¹, less than 5.0×10⁻⁴ s⁻¹, less than 6.0×10⁻⁴ s⁻¹, less than 7.0×10⁻⁴ s⁻¹, less than 8.0×10⁻⁴ s⁻¹, or less than 9.0×10⁻⁴ s⁻¹. In still other aspects of this embodiment, a targeting domain disclosed herein binds to its cognate receptor with a disassociation rate constant of, e.g., more than 1×10⁻³ s⁻¹, more than 1×10⁻⁴ s⁻¹, or more than 1×10⁻⁵ s⁻¹. In other aspects of this embodiment, a targeting domain disclosed herein binds to its cognate receptor with a disassociation rate constant of, e.g., more than 1.0×10⁻⁴ s⁻¹, more than 2.0×10⁻⁴ s⁻¹, more than 3.0×10⁻⁴ s⁻¹, more than 4.0×10⁻⁴ s⁻¹, more than 5.0×10⁻⁴ s⁻¹, more than 6.0×10⁻⁴ s⁻¹, more than 7.0×10⁻⁴ s⁻¹, more than 8.0×10⁻⁴ s⁻¹, or more than 9.0×10⁻⁴ s⁻¹.

In still another embodiment, a targeting domain disclosed herein has a disassociation rate constant that is less for its cognate target receptor relative to a non-cognate receptor. In other aspects of this embodiment, a targeting domain disclosed herein has a disassociation rate constant that is less for its cognate target receptor relative to a non-cognate receptor by, e.g., at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has a disassociation rate constant that is less for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10,000-fold, or about 10-fold to about 100,000-fold.

In another embodiment, a targeting domain disclosed herein has an equilibrium disassociation constant that confers preferential binding to its cognate receptor. In other aspects of this embodiment, a targeting domain disclosed herein binds to its cognate receptor with an equilibrium disassociation constant of, e.g., less than 0.500 nM. In yet other aspects of this embodiment, a targeting domain disclosed herein binds to its cognate receptor with an equilibrium disassociation constant of, e.g., less than 0.500 nM, less than 0.450 nM, less than 0.400 nM, less than 0.350 nM, less than 0.300 nM, less than 0.250 nM, less than 0.200 nM, less than 0.150 nM, less than 0.100 nM, or less than 0.050 nM. In other aspects of this embodiment, a targeting domain disclosed herein binds to its cognate receptor with an equilibrium disassociation constant of, e.g., more than 0.500 nM, more than 0.450 nM, more than 0.400 nM, more than 0.350 nM, more than 0.300 nM, more than 0.250 nM, more than 0.200 nM, more than 0.150 nM, more than 0.100 nM, or more than 0.050 nM.

In yet another embodiment, a targeting domain disclosed herein has an equilibrium disassociation constant that is greater for its cognate target receptor relative to a non-cognate receptor. In other aspects of this embodiment, a targeting domain disclosed herein has an equilibrium disassociation constant that is greater for its cognate target receptor relative to a non-cognate receptor by, e.g., at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has an equilibrium disassociation constant that is greater for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10,000-fold, or about 10-fold to about 100,000-fold.

In another embodiment, a targeting domain disclosed herein may be one that preferentially interacts with a receptor located on a sensory neuron. In an aspect of this embodiment, the sensory neuron targeting domain is one whose cognate receptor is located exclusively on the plasma membrane of sensory neurons. In another aspect of this embodiment, the sensory neuron targeting domain is one whose cognate receptor is located primarily on the plasma membrane of sensory neuron. For example, a receptor for a sensory neuron targeting domain is located primarily on a sensory neuron when, e.g., at least 60% of all cells that have a cognate receptor for a sensory neuron targeting domain on the surface of the plasma membrane are sensory neurons, at least 70% of all cells that have a cognate receptor for a sensory neuron targeting domain on the surface of the plasma membrane are sensory neurons, at least 80% of all cells that have a cognate receptor for a sensory neuron targeting domain on the surface of the plasma membrane are sensory neurons, or at least 90% of all cells that have a cognate receptor for a sensory neuron targeting domain on the surface of the plasma membrane are sensory neurons. In yet another aspect of this embodiment, the sensory neuron targeting domain is one whose cognate receptor is located on the plasma membrane of several types of cells, including sensory neurons. In still another aspect of this embodiment, the sensory neuron targeting domain is one whose cognate receptor is located on the plasma membrane of several types of cells, including sensory neurons, with the proviso that motor neurons are not one of the other types of cells.

In another embodiment, a targeting domain disclosed herein may be one that preferentially interacts with a receptor located on a sympathetic neuron. In an aspect of this embodiment, the sympathetic neuron targeting domain is one whose cognate receptor is located exclusively on the plasma membrane of sympathetic neurons. In another aspect of this embodiment, the sympathetic neuron targeting domain is one whose cognate receptor is located primarily on the plasma membrane of sympathetic neuron. For example, a receptor for a sympathetic neuron targeting domain is located primarily on a sympathetic neuron when, e.g., at least 60% of all cells that have a cognate receptor for a sympathetic neuron targeting domain on the surface of the plasma membrane are sympathetic neurons, at least 70% of all cells that have a cognate receptor for a sympathetic neuron targeting domain on the surface of the plasma membrane are sympathetic neurons, at least 80% of all cells that have a cognate receptor for a sympathetic neuron targeting domain on the surface of the plasma membrane are sympathetic neurons, or at least 90% of all cells that have a cognate receptor for a sympathetic neuron targeting domain on the surface of the plasma membrane are sympathetic neurons. In yet another aspect of this embodiment, the sympathetic neuron targeting domain is one whose cognate receptor is located on the plasma membrane of several types of cells, including sympathetic neurons. In still another aspect of this embodiment, the sympathetic neuron targeting domain is one whose cognate receptor is located on the plasma membrane of several types of cells, including sympathetic neurons, with the proviso that motor neurons are not one of the other types of cells.

In another embodiment, a targeting domain disclosed herein may be one that preferentially interacts with a receptor located on a parasympathetic neuron. In an aspect of this embodiment, the parasympathetic neuron targeting domain is one whose cognate receptor is located exclusively on the plasma membrane of parasympathetic neurons. In another aspect of this embodiment, the parasympathetic neuron targeting domain is one whose cognate receptor is located primarily on the plasma membrane of parasympathetic neuron. For example, a receptor for a parasympathetic neuron targeting domain is located primarily on a parasympathetic neuron when, e.g., at least 60% of all cells that have a cognate receptor for a parasympathetic neuron targeting domain on the surface of the plasma membrane are parasympathetic neurons, at least 70% of all cells that have a cognate receptor for a parasympathetic neuron targeting domain on the surface of the plasma membrane are parasympathetic neurons, at least 80% of all cells that have a cognate receptor for a parasympathetic neuron targeting domain on the surface of the plasma membrane are parasympathetic neurons, or at least 90% of all cells that have a cognate receptor for a parasympathetic neuron targeting domain on the surface of the plasma membrane are parasympathetic neurons. In yet another aspect of this embodiment, the parasympathetic neuron targeting domain is one whose cognate receptor is located on the plasma membrane of several types of cells, including parasympathetic neurons. In still another aspect of this embodiment, the parasympathetic neuron targeting domain is one whose cognate receptor is located on the plasma membrane of several types of cells, including parasympathetic neurons, with the proviso that motor neurons are not one of the other types of cells.

In another embodiment, a targeting domain disclosed herein is an opioid peptide targeting domain, a galanin peptide targeting domain, a PAR peptide targeting domain, a somatostatin peptide targeting domain, a neurotensin peptide targeting domain, a SLURP peptide targeting domain, an angiotensin peptide targeting domain, a tachykinin peptide targeting domain, a Neuropeptide Y related peptide targeting domain, a kinin peptide targeting domain, a melanocortin peptide targeting domain, or a granin peptide targeting domain, a glucagon like hormone peptide targeting domain, a secretin peptide targeting domain, a pituitary adenylate cyclase activating peptide (PACAP) peptide targeting domain, a growth hormone-releasing hormone (GHRH) peptide targeting domain, a vasoactive intestinal peptide (VIP) peptide targeting domain, a gastric inhibitory peptide (GIP) peptide targeting domain, a calcitonin peptide targeting domain, a visceral gut peptide targeting domain, a neurotrophin peptide targeting domain, a head activator (HA) peptide, a glial cell line-derived neurotrophic factor (GDNF) family of ligands (GFL) peptide targeting domain, a RF-amide related peptide (RFRP) peptide targeting domain, a neurohormone peptide targeting domain, or a neuroregulatory cytokine peptide targeting domain, an interleukin (IL) targeting domain, vascular endothelial growth factor (VEGF) targeting domain, an insulin-like growth factor (IGF) targeting domain, an epidermal growth factor (EGF) targeting domain, a Transformation Growth Factor-β (TGFβ) targeting domain, a Bone Morphogenetic Protein (BMP) targeting domain, a Growth and Differentiation Factor (GDF) targeting domain, an activin targeting domain, or a Fibroblast Growth Factor (FGF) targeting domain, or a Platelet-Derived Growth Factor (PDGF) targeting domain.

In an aspect of this embodiment, an opioid peptide targeting domain is an enkephalin peptide, a bovine adrenomedullary-22 (BAM22) peptide, an endomorphin peptide, an endorphin peptide, a dynorphin peptide, a nociceptin peptide, or a hemorphin peptide. In another aspect of this embodiment, an enkephalin peptide targeting domain is a Leu-enkephalin peptide, a Met-enkephalin peptide, a Met-enkephalin MRGL peptide, or a Met-enkephalin MRF peptide. In another aspect of this embodiment, a bovine adrenomedullary-22 peptide targeting domain is a BAM22 (1-12) peptide, a BAM22 (6-22) peptide, a BAM22 (8-22) peptide, or a BAM22 (1-22) peptide. In another aspect of this embodiment, an endomorphin peptide targeting domain is an endomorphin-1 peptide or an endomorphin-2 peptide. In another aspect of this embodiment, an endorphin peptide targeting domain an endorphin-αpeptide, a neoendorphin-α peptide, an endorphin-β peptide, a neoendorphin-β peptide, or an endorphin-γ peptide. In another aspect of this embodiment, a dynorphin peptide targeting domain is a dynorphin A peptide, a dynorphin B (leumorphin) peptide, or a rimorphin peptide. In another aspect of this embodiment, a nociceptin peptide targeting domain is a nociceptin RK peptide, a nociceptin peptide, a neuropeptide 1 peptide, a neuropeptide 2 peptide, or a neuropeptide 3 peptide. In another aspect of this embodiment, a hemorphin peptide targeting domain is a LVVH7 peptide, a VVH7 peptide, a VH7 peptide, a H7 peptide, a LVVH6 peptide, a LVVH5 peptide, a VVH5 peptide, a LVVH4 peptide, or a LVVH3 peptide.

In an aspect of this embodiment, a galanin peptide targeting domain is a galanin peptide, a galanin message-associated peptide (GMAP) peptide, a galanin like protein (GALP) peptide, or an alarin peptide.

In an aspect of this embodiment, a PAR peptide targeting domain is a PAR1 peptide, a PAR2 peptide, a PAR3 peptide and a PAR4 peptide. In an aspect of this embodiment, a somatostatin peptide targeting domain is a somatostatin peptide or a cortistatin peptide. In an aspect of this embodiment, a neurotensin peptide targeting domain a neurotensin or a neuromedin N. In an aspect of this embodiment, a SLURP peptide targeting domain is a SLURP-1 peptide or a SLURP-2 peptide. In an aspect of this embodiment, an angiotensin peptide targeting domain is an angiotensin peptide.

In an aspect of this embodiment, a tachykinin peptide targeting domain is a Substance P peptide, a neuropeptide K peptide, a neuropeptide gamma peptide, a neurokinin A peptide, a neurokinin B peptide, a hemokinin peptide, or a endokinin peptide. In an aspect of this embodiment, a Neuropeptide Y related peptide targeting domain is a Neuropeptide Y peptide, a Peptide YY peptide, Pancreatic peptide peptide, a Pancreatic icosapeptide peptide, a Pancreatic Hormone domain peptide, a CXCL12 peptide, and a Sjogren syndrome antigen B peptide. In an aspect of this embodiment, a kinin peptide targeting domain is a bradykinin peptide, a kallidin peptide, a desArg9 bradykinin peptide, a desArg10 bradykinin peptide, a kininogen peptide, gonadotropin releasing hormone 1 peptide, chemokine peptide, an arginine vasopressin peptide.

In an aspect of this embodiment, a melanocortin peptide targeting domain comprises a melanocyte stimulating hormone peptide, an adrenocorticotropin peptide, a lipotropin peptide, or a melanocortin peptide derived neuropeptide. In an aspect of this embodiment, a melanocyte stimulating hormone peptide targeting domain comprises an α-melanocyte stimulating hormone peptide, a β-melanocyte stimulating hormone peptide, or a γ-melanocyte stimulating hormone peptide. In an aspect of this embodiment, an adrenocorticotropin peptide targeting domain comprises an adrenocorticotropin or a Corticotropin-like intermediary peptide. In an aspect of this embodiment, a lipotropin peptide targeting domain comprises a β-lipotropin peptide or a γ-lipotropin peptide.

In an aspect of this embodiment, a granin peptide targeting domain comprises a chromogranin A peptide, a chromogranin B peptide, a chromogranin C (secretogranin II) peptide, a secretogranin IV peptide, or a secretogranin VI peptide. In an aspect of this embodiment, a chromogranin A peptide targeting domain comprises a β-granin peptide, a vasostatin peptide, a chromostatin peptide, a pancreastatin peptide, a WE-14 peptide, a catestatin peptide, a parastatin peptide, or a GE-25 peptide. In an aspect of this embodiment, a chromogranin B peptide targeting domain comprises a GAWK peptide, an adrenomedullary peptide, or a secretolytin peptide. In an aspect of this embodiment, a chromogranin C peptide targeting domain comprises a secretoneurin peptide.

In an aspect of this embodiment, a glucagons-like hormone peptide targeting domain is a glucagon-like peptide-1, a glucagon-like peptide-2, a glicentin, a glicentin-related peptide (GRPP), a glucagon, or an oxyntomodulin (OXY). In an aspect of this embodiment, a secretin peptide targeting domain is a secretin peptide. In an aspect of this embodiment, a pituitary adenylate cyclase activating peptide targeting domain is a pituitary adenylate cyclase activating peptide. In an aspect of this embodiment, a growth hormone-releasing hormone peptide targeting domain a growth hormone-releasing hormone peptide. In an aspect of this embodiment, a vasoactive intestinal peptide targeting domain is a vasoactive intestinal peptide-1 peptide or a vasoactive intestinal peptide-2 peptide. In an aspect of this embodiment, a gastric inhibitory peptide targeting domain is a gastric inhibitory peptide. In an aspect of this embodiment, a calcitonin peptide targeting domain is a calcitonin peptide, an amylin peptide, a calcitonin-related peptide a, a calcitonin-related peptide β, and a islet amyloid peptide. In an aspect of this embodiment, a visceral gut peptide targeting domain is a gastrin peptide, a gastrin-releasing peptide, or a cholecystokinin peptide.

In an aspect of this embodiment, a neurotrophin peptide targeting domain is a nerve growth factor (NGF) peptide, a brain derived neurotrophic factor (BDNF) peptide, a neurotrophin-3 (NT-3) peptide, a neurotrophin-4/5 (NT-4/5) peptide, or an amyloid beta (A4) precursor protein neurotrophin (APP) peptide. In an aspect of this embodiment, a head activator peptide targeting domain is a head activator peptide. In an aspect of this embodiment, a glial cell line-derived neurotrophic factor family of ligands peptide targeting domain is a glial cell line-derived neurotrophic factor peptide, a Neurturin peptide, a Persephrin peptide, or an Artemin peptide. In an aspect of this embodiment, a RF-amide related peptide targeting domain a RF-amide related peptide-1, a RF-amide related peptide-2, a RF-amide related peptide-3, a neuropeptide AF, or a neuropeptide FF.

In an aspect of this embodiment, a neurohormone peptide targeting domain is a corticotropin-releasing hormone (CCRH), a parathyroid hormone (PTH), a parathyroid hormone-like hormone (PTHLH), a PHYH, a thyrotropin-releasing hormone (TRH), an urocortin-1 (UCN1), an urocortin-2 (UCN2), an urocortin-3 (UCN3), or an urotensin 2 (UTS2). In an aspect of this embodiment, a neuroregulatory cytokine peptide targeting domain is a ciliary neurotrophic factor peptide, a glycophorin-A peptide, a leukemia inhibitory factor peptide, a cardiotrophin-1 peptide, a cardiotrophin-like cytokine peptide, a neuroleukin peptide, and an onostatin M peptide. In an aspect of this embodiment, an IL peptide targeting domain is an IL-1 peptide, an IL-2 peptide, an IL-3 peptide, an IL-4 peptide, an IL-5 peptide, an IL-6 peptide, an IL-7 peptide, an IL-8 peptide, an IL-9 peptide, an IL-10 peptide, an IL-11 peptide, an IL-12 peptide, an IL-18 peptide, an IL-32 peptide, or an IL-33 peptide.

In an aspect of this embodiment, a VEGF peptide targeting domain is a VEGF-A peptide, a VEGF-B peptide, a VEGF-C peptide, a VEGF-D peptide, or a placenta growth factor (PIGF) peptide. In an aspect of this embodiment, an IGF peptide targeting domain is an IGF-1 peptide or an IGF-2 peptide. In an aspect of this embodiment, an EGF peptide targeting domain an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4). In an aspect of this embodiment, a FGF peptide targeting domain is a FGF1 peptide, a FGF2 peptide, a FGF3 peptide, a FGF4 peptide, a FGF5 peptide, a FGF6 peptide, a FGF7 peptide, a FGF8 peptide, a FGF9 peptide, a FGF10 peptide, a FGF17 peptide, or a FGF18 peptide. In an aspect of this embodiment, a PDGF peptide targeting domain is a PDGFα peptide or a PDGFβ peptide.

In an aspect of this embodiment, a TGFβ peptide targeting domain is a TGFβ1 peptide, a TGFβ2 peptide, a TGFβ3 peptide, or a TGFβ4 peptide. In an aspect of this embodiment, a BMP peptide targeting domain is a BMP2 peptide, a BMP3 peptide, a BMP4 peptide, a BMP5 peptide, a BMP6 peptide, a BMP7 peptide, a BMP8 peptide, or a BMP10 peptide. In an aspect of this embodiment, a GDF peptide targeting domain is a GDF1 peptide, a GDF2 peptide, a GDF3 peptide, a GDF5 peptide, a GDF6 peptide, a GDF7 peptide, a GDF8 peptide, a GDF10 peptide, a GDF11 peptide, or a GDF15 peptide. In an aspect of this embodiment, an activin peptide targeting domain is an activin A peptide, an activin B peptide, an activin C peptide, an activin E peptide, or an inhibin A peptide.

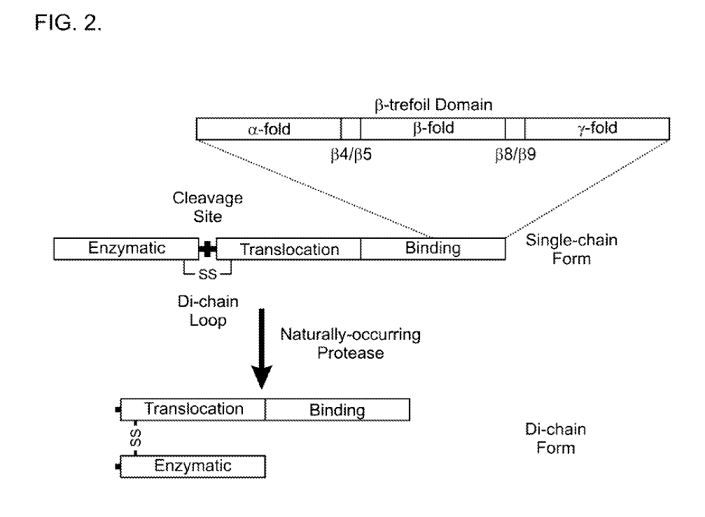

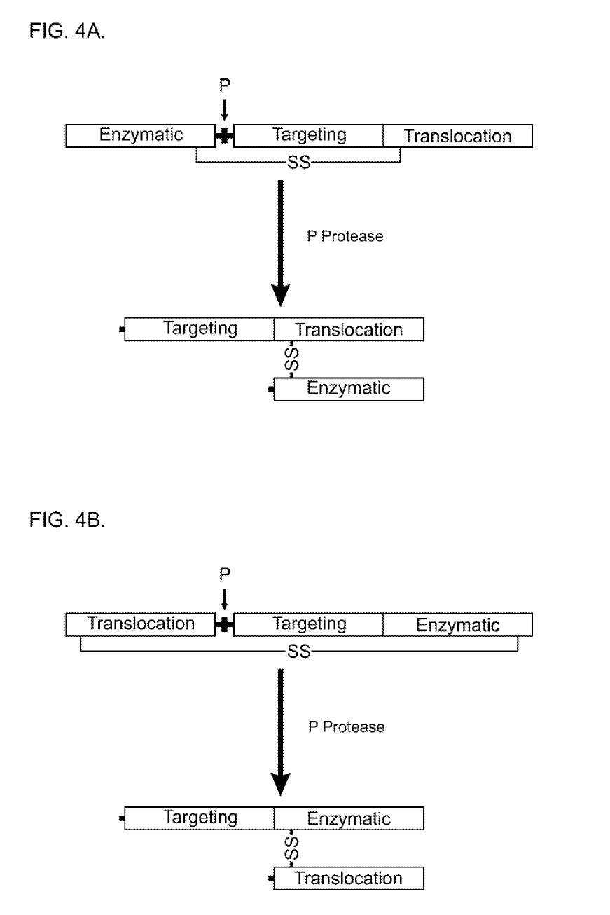

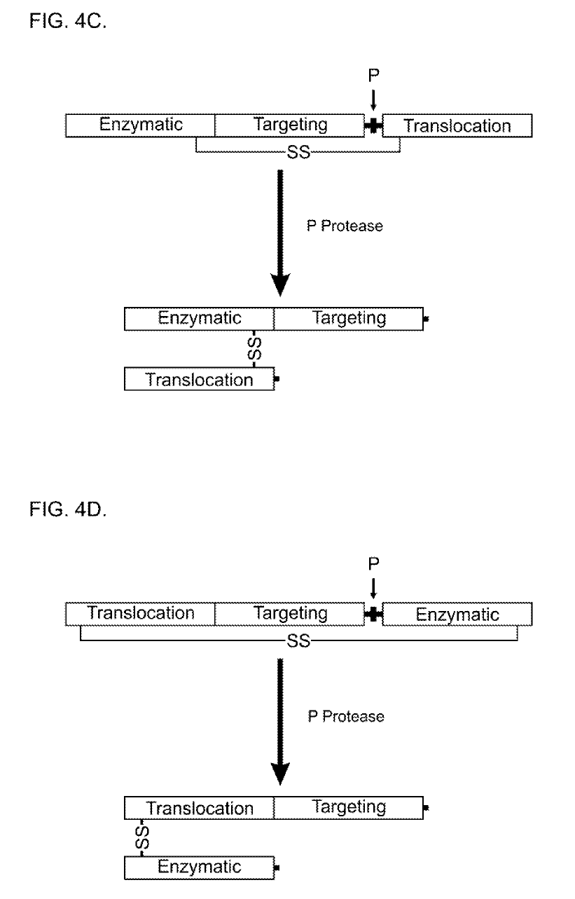

As discussed above, naturally-occurring Clostridial toxins are organized into three functional domains comprising a linear amino-to-carboxyl single polypeptide order of the enzymatic domain (amino region position), the translocation domain (middle region position) and the binding domain (carboxyl region position) (FIG. 2). This naturally-occurring order can be referred to as the carboxyl presentation of the binding domain because the domain necessary for binding to the receptor is located at the carboxyl region position of the Clostridial toxin. However, it has been shown that Clostridial toxins can be modified by rearranging the linear amino-to-carboxyl single polypeptide order of the three major domains and locating a targeting moiety at the amino region position of a Clostridial toxin, referred to as amino presentation, as well as in the middle region position, referred to as central presentation (FIG. 4).

Thus, a TEM can comprise a targeting domain in any and all locations with the proviso that TEM is capable of performing the intoxication process. Non-limiting examples include, locating a targeting domain at the amino terminus of a TEM; locating a targeting domain between a Clostridial toxin enzymatic domain and a Clostridial toxin translocation domain of a TEM; and locating a targeting domain at the carboxyl terminus of a TEM. Other non-limiting examples include, locating a targeting domain between a Clostridial toxin enzymatic domain and a Clostridial toxin translocation domain of a TEM. The enzymatic domain of naturally-occurring Clostridial toxins contains the native start methionine. Thus, in domain organizations where the enzymatic domain is not in the amino-terminal location an amino acid sequence comprising the start methionine should be placed in front of the amino-terminal domain. Likewise, where a targeting domain is in the amino-terminal position, an amino acid sequence comprising a start methionine and a protease cleavage site may be operably-linked in situations in which a targeting domain requires a free amino terminus, see, e.g., Shengwen Li et al., Degradable Clostridial Toxins, U.S. patent application Ser. No. 11/572,512 (Jan. 23, 2007), which is hereby incorporated by reference in its entirety. In addition, it is known in the art that when adding a polypeptide that is operably-linked to the amino terminus of another polypeptide comprising the start methionine that the original methionine residue can be deleted.

A TEM disclosed herein may optionally comprise an exogenous protease cleavage site that allows the use of an exogenous protease to convert the single-chain polypeptide form of a TEM into its more active di-chain form. As used herein, the term “exogenous protease cleavage site” is synonymous with a “non-naturally occurring protease cleavage site” or “non-native protease cleavage site” and means a protease cleavage site that is not naturally found in a di-chain loop region from a naturally occurring Clostridial toxin.

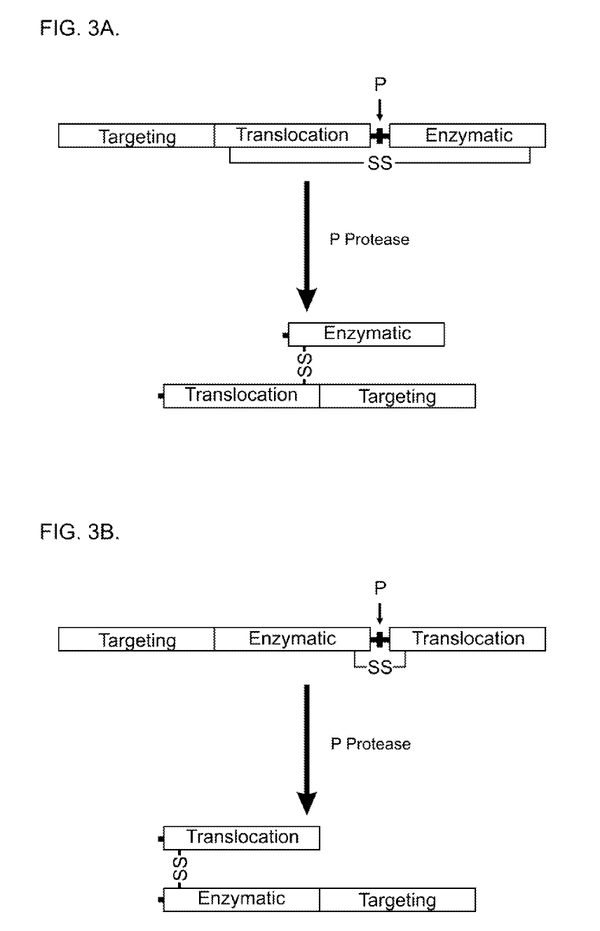

Naturally-occurring Clostridial toxins are each translated as a single-chain polypeptide of approximately 150 kDa that is subsequently cleaved by proteolytic scission within a disulfide loop by a naturally-occurring protease (FIG. 2). This cleavage occurs within the discrete di-chain loop region located between two cysteine residues that form a disulfide bridge and comprising an endogenous protease cleavage site. As used herein, the term “endogenous di-chain loop protease cleavage site” is synonymous with a “naturally occurring di-chain loop protease cleavage site” and refers to a naturally occurring protease cleavage site found within the di-chain loop region of a naturally occurring Clostridial toxin. This posttranslational processing yields a di-chain molecule comprising an approximately 50 kDa light chain, comprising the enzymatic domain, and an approximately 100 kDa heavy chain, comprising the translocation and cell binding domains, the light chain and heavy chain being held together by the single disulfide bond and non-covalent interactions (FIG. 2). Recombinantly-produced Clostridial toxins generally substitute the naturally-occurring di-chain loop protease cleavage site with an exogenous protease cleavage site to facilitate production of a recombinant di-chain molecule (FIGS. 3-5). See e.g., Dolly, J. O. et al., Activatable Clostridial Toxins, U.S. Pat. No. 7,419,676 (Sep. 2, 2008), which is hereby incorporated by reference.

Although TEMs vary in their overall molecular weight because the size of the targeting domain, the activation process and its reliance on an exogenous cleavage site is essentially the same as that for recombinantly-produced Clostridial toxins. See e.g., Steward, et al., Activatable Clostridial Toxins, US 2009/0081730; Steward, et al., Modified Clostridial Toxins with Enhanced Translocation Capabilities and Altered Targeting Activity For Non-Clostridial Toxin Target Cells, U.S. patent application Ser. No. 11/776,075; Steward, et al., Modified Clostridial Toxins with Enhanced Translocation Capabilities and Altered Targeting Activity for Clostridial Toxin Target Cells, US 2008/0241881, each of which is hereby incorporated by reference. In general, the activation process that converts the single-chain polypeptide into its di-chain form using exogenous proteases can be used to process TEMs having a targeting domain organized in an amino presentation, central presentation, or carboxyl presentation arrangement. This is because for most targeting domains the amino-terminus of the moiety does not participate in receptor binding. As such, a wide range of protease cleavage sites can be used to produce an active di-chain form of a TEM. However, targeting domains requiring a free amino-terminus for receptor binding require a protease cleavage site whose scissile bond is located at the carboxyl terminus. The use of protease cleavage site is the design of a TEM are described in, e.g., Steward, et al., Activatable Clostridial toxins, US 2009/0069238; Ghanshani, et al., Modified Clostridial Toxins Comprising an Integrated Protease Cleavage Site-Binding Domain, US 2011/0189162; and Ghanshani, et al., Methods of Intracellular Conversion of Single-Chain Proteins into their Di-chain Form, International Patent Application Serial No. PCT/US2011/22272, each of which is incorporated by reference in its entirety.

Non-limiting examples of exogenous protease cleavage sites include, e.g., a plant papain cleavage site, an insect papain cleavage site, a crustacian papain cleavage site, an enterokinase protease cleavage site, a Tobacco Etch Virus protease cleavage site, a Tobacco Vein Mottling Virus protease cleavage site, a human rhinovirus 3C protease cleavage site, a human enterovirus 3C protease cleavage site, a subtilisin cleavage site, a hydroxylamine cleavage site, a SUMO/ULP-1 protease cleavage site, and a Caspase 3 cleavage site.

Thus, in an embodiment, a TEM can comprise an amino to carboxyl single polypeptide linear order comprising a targeting domain, a translocation domain, an exogenous protease cleavage site and an enzymatic domain (FIG. 3A). In an aspect of this embodiment, a TEM can comprise an amino to carboxyl single polypeptide linear order comprising a targeting domain, a Clostridial toxin translocation domain, an exogenous protease cleavage site and a Clostridial toxin enzymatic domain.

In another embodiment, a TEM can comprise an amino to carboxyl single polypeptide linear order comprising a targeting domain, an enzymatic domain, an exogenous protease cleavage site, and a translocation domain (FIG. 3B). In an aspect of this embodiment, a TEM can comprise an amino to carboxyl single polypeptide linear order comprising a targeting domain, a Clostridial toxin enzymatic domain, an exogenous protease cleavage site, a Clostridial toxin translocation domain.

In yet another embodiment, a TEM can comprise an amino to carboxyl single polypeptide linear order comprising an enzymatic domain, an exogenous protease cleavage site, a targeting domain, and a translocation domain (FIG. 4A). In an aspect of this embodiment, a TEM can comprise an amino to carboxyl single polypeptide linear order comprising a Clostridial toxin enzymatic domain, an exogenous protease cleavage site, a targeting domain, and a Clostridial toxin translocation domain.

In yet another embodiment, a TEM can comprise an amino to carboxyl single polypeptide linear order comprising a translocation domain, an exogenous protease cleavage site, a targeting domain, and an enzymatic domain (FIG. 4B). In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

In another embodiment, a TEM can comprise an amino to carboxyl single polypeptide linear order comprising an enzymatic domain, a targeting domain, an exogenous protease cleavage site, and a translocation domain (FIG. 4C). In an aspect of this embodiment, a TEM can comprise an amino to carboxyl single polypeptide linear order comprising a Clostridial toxin enzymatic domain, a targeting domain, an exogenous protease cleavage site, a Clostridial toxin translocation domain.

In yet another embodiment, a TEM can comprise an amino to carboxyl single polypeptide linear order comprising a translocation domain, a targeting domain, an exogenous protease cleavage site and an enzymatic domain (FIG. 4D). In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

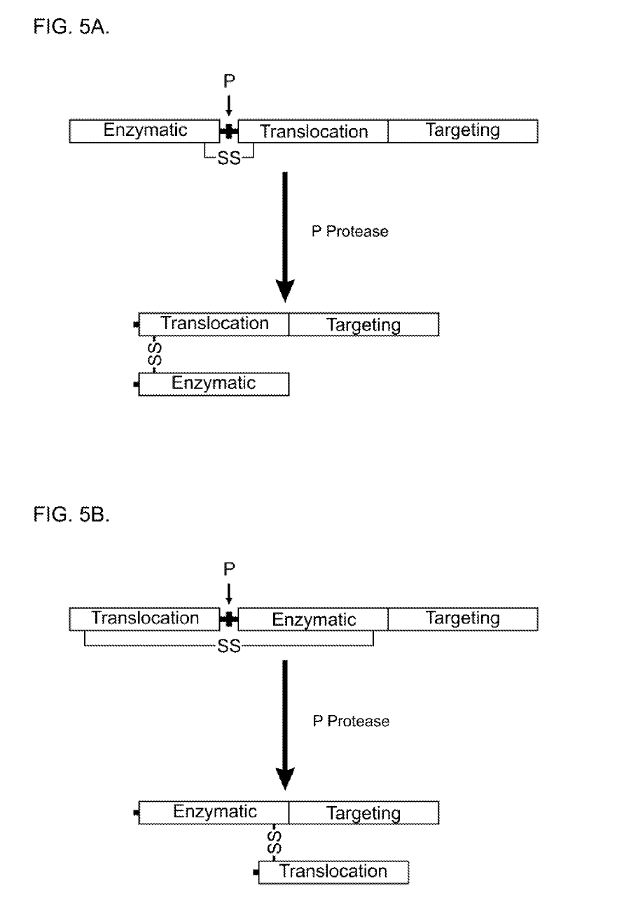

In still another embodiment, a TEM can comprise an amino to carboxyl single polypeptide linear order comprising an enzymatic domain, an exogenous protease cleavage site, a translocation domain, and a targeting domain (FIG. 5A). In an aspect of this embodiment, a TEM can comprise an amino to carboxyl single polypeptide linear order comprising a Clostridial toxin enzymatic domain, an exogenous protease cleavage site, a Clostridial toxin translocation domain, and a targeting domain.

In still another embodiment, a TEM can comprise an amino to carboxyl single polypeptide linear order comprising a translocation domain, an exogenous protease cleavage site, an enzymatic domain and a targeting domain, (FIG. 5B). In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

Non-limiting examples of TEMs disclosed herein, including TEMs comprising a Clostridal toxin enzymatic domain, a Clostridial toxin translocation domain and a targeting domain, the use of an exogenous protease cleavage site, and the design of amino presentation, central presentation and carboxyl presentation TEMs are described in, e.g., U.S. Pat. No. 7,959,933, Activatable Recombinant Neurotoxins, U.S. Pat. No. 7,897,157, Activatable Clostridial Toxins; U.S. Pat. No. 7,833,535, Clostridial Toxin Derivatives and Methods for Treating Pain; U.S. Pat. No. 7,811,584, Multivalent Clostridial Toxins; U.S. Pat. No. 7,780,968, Clostridial Toxin Derivatives and Methods for Treating Pain; U.S. Pat. No. 7,749,514, Activatable Clostridial Toxins, U.S. Pat. No. 7,740,868, Activatable Clostridial Toxins; U.S. Pat. No. 7,736,659, Clostridial Toxin Derivatives and Methods for Treating Pain; U.S. Pat. No. 7,709,228, Activatable Recombinant Neurotoxins; U.S. Pat. No. 7,704,512, Clostridial Toxin Derivatives and Methods for Treating Pain; U.S. Pat. No. 7,659,092, Fusion Proteins; U.S. Pat. No. 7,658,933, Non-Cytotoxic Protein Conjugates; U.S. Pat. No. 7,622,127, Clostridial Toxin Derivatives and Methods for Treating Pain; U.S. Pat. No. 7,514,088, Multivalent Clostridial Toxin Derivatives and Methods of Their Use; U.S. Pat. No. 7,425,338, Clostridial Toxin Derivatives and Methods for Treating Pain; U.S. Pat. No. 7,422,877, Activatable Recombinant Neurotoxins; U.S. Pat. No. 7,419,676, Activatable Recombinant Neurotoxins; U.S. Pat. No. 7,413,742, Clostridial Toxin Derivatives and Methods for Treating Pain; U.S. Pat. No. 7,262,291, Clostridial Toxin Derivatives and Methods for Treating Pain; U.S. Pat. No. 7,244,437, Clostridial Toxin Derivatives and Methods for Treating Pain; U.S. Pat. No. 7,244,436, Clostridial Toxin Derivatives and Methods for Treating Pain; U.S. Pat. No. 7,138,127, Clostridial Toxin Derivatives and Methods for Treating Pain; U.S. Pat. No. 7,132,259, Activatable Recombinant Neurotoxins; U.S. Pat. No. 7,056,729, Botulinum Neurotoxin-Substance P Conjugate or Fusion Protein for Treating Pain; U.S. Pat. No. 6,641,820, Clostridial Toxin Derivatives and Methods to Treat Pain; U.S. Pat. No. 6,500,436, Clostridial Toxin Derivatives and Methods for Treating Pain; US 2011/0091437, Fusion Proteins; US 2011/0070621, Multivalent Clostridial Toxins; US 2011/0027256, Fusion Proteins; US 2010/0247509, Fusion Proteins; US 2010/0041098, Modified Clostridial Toxins with Altered Targeting Capabilities for Clostridial Toxin Target Cells; US 2010/0034802, Treatment of Pain; US 2009/0162341, Non-Cytotoxic Protein Conjugates; US 2009/0087458, Activatable Recombinant Neurotoxins; US 2009/0081730, Activatable Recombinant Neurotoxins; US 2009/0069238, Activatable Clostridial Toxins; US 2009/0042270, Activatable Recombinant Neurotoxins; US 2009/0030182, Activatable Recombinant Neurotoxins; US 2009/0018081, Activatable Clostridial Toxins; US 2009/0005313, Activatable Clostridial Toxins; US 2009/0004224, Activatable Clostridial Toxins; US 2008/0317783, Clostridial Toxin Derivatives and Methods for Treating Pain; US 2008/0241881, Modified Clostridial Toxins with Enhanced Translocation Capabilities and Altered Targeting Activity for Clostridial Toxin Target Cells; WO 2006/099590, Modified Clostridial Toxins with Altered Targeting Capabilities for Clostridial Toxin Target Cells; WO 2006/101809, Modified Clostridial Toxins with Enhanced Targeting Capabilities for Endogenous Clostridial Toxin Receptor Systems; WO 2007/106115, Modified Clostridial Toxins with Altered Targeting Capabilities for Clostridial Toxin Target Cells; WO 2008/008803, Modified Clostridial Toxins with Enhanced Translocation Capabilities and Altered Targeting Activity for Clostridial Toxin Target Cells; WO 2008/008805, Modified Clostridial Toxins with Enhanced Translocation Capabilities and Altered Targeting Activity For Non-Clostridial Toxin Target Cells; WO 2008/105901, Modified Clostridial Toxins with Enhanced Translocation Capability and Enhanced Targeting Activity; WO 2011/020052, Methods of Treating Cancer Using Opioid Retargeted Endpeptidases; WO 2011/020056, Methods of Treating Cancer Using Galanin Retargeted Endpeptidases; WO 2011/020114, Methods of Treating Cancer Using Tachykinin Retargeted Endopeptidases; WO 2011/020115, Methods of Treating Cancer Using Growth Factor Retargeted Endopeptidases; WO 2011/020117, Methods of Treating Cancer Using Neurotrophin Retargeted Endopeptidases; WO 2011/020119, Methods of Treating Cancer Using Glucagon-Like Hormone Retargeted Endopeptidases; each of which is incorporated by reference in its entirety.

Aspects of the present specification disclose, in part, a composition. In one aspect of this embodiment, a composition comprises a TEM as disclosed herein. In another aspect of this embodiment, a composition comprises a Clostridial toxin and a TEM as disclosed herein. Any of the compositions disclosed herein can be useful in a method of treating disclosed herein, with the proviso that the composition prevents or reduces a symptom associated with condition being treated. A Clostridial toxin and a TEM as disclosed herein may be provided as separate compositions or as part of a single composition. It is also understood that the two or more different Clostridial toxins and/or TEMs can be provided as separate compositions or as part of a single composition.

A composition disclosed herein is generally administered as a pharmaceutical acceptable composition. As used herein, the term “pharmaceutically acceptable” means any molecular entity or composition that does not produce an adverse, allergic or other untoward or unwanted reaction when administered to an individual. As used herein, the term “pharmaceutically acceptable composition” is synonymous with “pharmaceutical composition” and means a therapeutically effective concentration of an active ingredient, such as, e.g., any of the Clostridial toxins and/or TEMs disclosed herein. A pharmaceutical composition disclosed herein is useful for medical and veterinary applications. A pharmaceutical composition may be administered to an individual alone, or in combination with other supplementary active ingredients, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

A pharmaceutical composition disclosed herein may optionally include a pharmaceutically acceptable carrier that facilitates processing of an active ingredient into pharmaceutically acceptable compositions. As used herein, the term “pharmacologically acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active ingredient, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN'S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003). These protocols are routine procedures and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition. Exemplary pharmaceutical composition comprising a TEM are described in Hunt, et al., Animal Protein-Free Pharmaceutical Compositions, US Ser. No. 12/331,816; and Dasari, et al., Clostridial Toxin Pharmaceutical Compositions, WO/2010/090677, each of which is hereby incorporated by reference in its entirety.

In an embodiment, a composition is a pharmaceutical composition comprising a TEM. In aspects of this embodiment, a pharmaceutical composition comprising a TEM further comprises a pharmacological carrier, a pharmaceutical component, or both a pharmacological carrier and a pharmaceutical component. In other aspects of this embodiment, a pharmaceutical composition comprising a TEM further comprises at least one pharmacological carrier, at least one pharmaceutical component, or at least one pharmacological carrier and at least one pharmaceutical component.

In another embodiment, a composition is a pharmaceutical composition comprising a Clostridial toxin. In aspects of this embodiment, a pharmaceutical composition comprising a Clostridial toxin further comprises a pharmacological carrier, a pharmaceutical component, or both a pharmacological carrier and a pharmaceutical component. In other aspects of this embodiment, a pharmaceutical composition comprising a Clostridial toxin further comprises at least one pharmacological carrier, at least one pharmaceutical component, or at least one pharmacological carrier and at least one pharmaceutical component.

In yet another embodiment, a composition is a pharmaceutical composition comprising a Clostridial toxin and a TEM. In aspects of this embodiment, a pharmaceutical composition comprising a Clostridial toxin and a TEM further comprises a pharmacological carrier, a pharmaceutical component, or both a pharmacological carrier and a pharmaceutical component. In other aspects of this embodiment, a pharmaceutical composition comprising a Clostridial toxin and a TEM further comprises at least one pharmacological carrier, at least one pharmaceutical component, or at least one pharmacological carrier and at least one pharmaceutical component.

Aspects of the present specification disclose, in part, treating an individual suffering from an involuntary movement disorder. As used herein, the term “treating,” refers to reducing or eliminating in an individual a clinical symptom of an involuntary movement disorder; or delaying or preventing in an individual the onset of a clinical symptom of an involuntary movement disorder. For example, the term “treating” can mean reducing a symptom of a condition characterized by an involuntary movement disorder by, e.g., at least 20%, at least 30%, at least 40%, at least 50%, at least 60%, at least 70%, at least 80%, at least 90% or at least 100%. The actual symptoms associated with an involuntary movement disorder are well known and can be determined by a person of ordinary skill in the art by taking into account factors, including, without limitation, the location of the involuntary movement disorder, the cause of the involuntary movement disorder, the severity of the involuntary movement disorder, and/or the tissue or organ affected by the involuntary movement disorder. Those of skill in the art will know the appropriate symptoms or indicators associated with specific sensory disturbance disorder and will know how to determine if an individual is a candidate for treatment as disclosed herein.

Aspects of the present specification disclose, in part, treating an individual suffering from an involuntary movement disorder. As used herein, the term “treating,” refers to reducing or eliminating in an individual a clinical symptom of an involuntary movement disorder; or delaying or preventing in an individual the onset of a clinical symptom of an involuntary movement disorder. For example, the term “treating” can mean reducing a symptom of a condition characterized by an involuntary movement disorder by, e.g., at least 20%, at least 30%, at least 40%, at least 50%, at least 60%, at least 70%, at least 80%, at least 90% or at least 100%. The actual symptoms associated with an involuntary movement disorder are well known and can be determined by a person of ordinary skill in the art by taking into account factors, including, without limitation, the location of the involuntary movement disorder, the cause of the involuntary movement disorder, the severity of the involuntary movement disorder, and/or the tissue or organ affected by the involuntary movement disorder. Those of skill in the art will know the appropriate symptoms or indicators associated with specific involuntary movement disorder and will know how to determine if an individual is a candidate for treatment as disclosed herein.

As used herein, the term “involuntary movement disorder” refers to an involuntary movement disorder where at least one of the underlying symptoms being treated is due to a sensory nerve-based etiology, a sympathetic nerve-based etiology, and/or a parasympathetic nerve-based etiology. Typically such etiologies will involve an abnormal overactivity of a nerve that results in symptoms of an involuntary movement disorder, or any normal activity of a nerve that needs to be reduced or stopped for a period of time in order to treat an involuntary movement disorder. Involuntary movement disorders include, without limitation, an ataxia, a chorea, a dyskinesia, a synkinesia, a dystonia, a cerebral palsy, a periodic limb movement disorder, a restless leg syndrome, a spasm, a stereotypy, a tic, and a tremor.

Ataxia (or dystaxia) refers to an involuntary movement disorder where an individual exhibits a gross lack of coordination of muscle movements. Ataxia is a manifestation of neurological dysfunction due to any of a variety of causes. One example is Wilson's disease, in which toxic levels of copper accumulate in the brain and thus lead to various neurological symptoms including ataxia, but also tremors, dystonia, the rigidity of posture seen in individuals with Parkinson's. As another example, Ataxia telangiectasia (also known as Boder-Sedgwick or Louis-Bar syndrome), is an inherited neurodegenerative disease in which affected subjects manifest ataxia. Ataxia can also be classified as, without limitation, cerebellar, sensory, or vestibular ataxia.

Cerebellar ataxia is ataxia due to dysfunction of the cerebellum, and can manifest in a variety of ways depending on what part of the cerebellum is dysfunctional. Cerebellar ataxia may arise from dysfunction in, for example, the cerebrocerebellum, spinocerebellum, or vestibulocerebellum. Cerebellar ataxia arising from dysfunction in the cerebrocerebellum results in disturbances in intentional movements in the extremities (appendicular ataxia). These include intention tremors, abnormalities in writing, dysarthria (slurred speech or variations in voice intensity), dysdiadochokinesia (inability to perform rapid, alternating movements), dysmetria (lack of ability to gauge distance), and loss of check reflex. Cerebellar ataxia arising from dysfunction in the vestibulocerebellum results in impaired balance and control of eye movements. Cerebellar ataxia arising from dysfunction in the spinocerebellum results in an abnormal gait (truncal ataxia).

Sensory ataxia is ataxia manifesting as proprioception (reduced ability to gauge the positions of body parts), evidenced by an abnormal gait and unstable posture. It can be caused by dysfunction of the dorsal column of the spinal cord or certain regions of the brain, including the cerebellum, parietal lobes and thalamus; or, it can be caused by peripheral neuropathies.

Vestibular ataxia is ataxia due to dysfunction of the vestibular system, which manifest as disequilibrium and can be accompanied by vertigo, nausea and vomiting.

Chorea (or choreia) refers to an involuntary movement disorder where an individual displays rapid, involuntary movements that are not repetitive or rhythmic. Chorea can arise from or be a complication of a variety of conditions and disorders, including, without limitation: rheumatic fever (where it is known as Sydenham's chorea, chorea minor, rheumatic chorea, or St. Vitus Dance); Huntington's disease, which is a genetic disorder; pregnancy, with which chorea is a rare complication (where it is termed chorea gravidarum); Wilson's disease (a genetic disorder leading to the accumulation of toxic levels of copper in various tissues, mainly the liver and brain); drug use; McLeod syndrome, a feature of which is limb chorea (as well as facial tics, other involuntary oral movements, seizures, etc.); metabolic and/or endocrine-related disorders; and, vascular incidents. Ballism is a more serious form of chorea, where an individual exhibits violent, thrashing movements, and often a peculiar gait and odd postures as well. Hemiballismus is ballism affecting only one side of the body.

A dyskinesia refers to an involuntary movement disorder where an individual displays involuntary movements and diminished voluntary movements. Manifestations of involuntary movements due to dyskinesia include, without limitation: tremor, such as in the hands; tics; chorea; and uncontrollable movement of the upper body or lower extremities. A lack of coordination of internal muscles may also occur, such as in the respiratory muscles. Dyskinesia manifests as a symptom of various medical disorders.

Tardive dyskinesia (TD) is a particular form of dyskinesia, so named because it has a slow or late (tardive) onset, which is difficult to treat and generally follows the use of antipsychotic drugs or drugs for the treatment of gastrointestinal disorders. Individuals with TD exhibit rapid involuntary movements such as, for example, blinking, grimacing, sticking out the tongue, and smacking or puckering the lips. The involuntary movements are not limited to the face, however, and may occur in the extremities. It has been said that subjects with TD have difficulty in not moving. Variants of TD include, without limitation: tardive akathisia, which manifests as an irresistible urge to move the body; tardive dystonia, similar to standard dystonia but permanent; tardive myoclonus, which manifests as quick jerks of muscles in the body, extremities, face, and neck; and, tardive tourettism, which is a tic similar to Tourette Syndrome but differing in the details of onset. The causes of TD are not entirely known, but appear to be related to dopamine supersensitivity as a result of treatment with neuroleptics.

A synkinesia refers to an involuntary movement disorder where an individual displays involuntary movements that accompany voluntary movements; for example, voluntarily smiling may induce an involuntary squint in the eye of a subject. Synkinesia may also be referred to as associated movements or mirror movements. Synkinesia can be caused by nerve damage following trauma. For example, facial synkinesia can be a sequela to idiopathic facial nerve paralysis (Bell's Palsy or Facial Palsy). It is also a symptom of some congenital disorders, such as Duane-Retraction Syndrome and Marcus Gunn phenomenon.

A dystonia refers to an involuntary movement disorder where an individual has sustained muscle contractions usually producing twisting, jerking, and/or repetitive movements of the body or a body part or abnormal postures or positions of the body or a body part. Almost all dystonic movements share a directional quality that is typically sustained, sometimes for an instant, as well as a consistency and predictability Dystonia movements are directional, forcing the involved body part or region into an abnormal position, which is consistently present. These neurological-based movement disorders may be hereditary or caused by other factors such as birth-related or other physical trauma, infection, poisoning (e.g., lead poisoning) or reaction to pharmaceutical drugs, particularly neuroleptics.

Dystonia may occur as a primary condition (idiopathic dystonia) that is familial or occurs in the absence of a family history. It may result from certain environmental factors or “insults” that affect the brain (secondary or symptomatic dystonia). Dystonia may be associated with certain nondegenerative, neurochemical disorders (known as “dystonia-plus syndromes”) that are characterized by neurologic features, such as parkinsonism or myoclonus. Dystonia is also a primary feature of certain, usually hereditary, neurodegenerative disorders (so-called “heredodegenerative dystonias”). One example is Wilson's disease, in which neurological damage results in various symptoms including dystonia. A dystonia usually begins in a single body part. It may either remain restricted to that area or spread to involve another region or regions.

Dystonic movements are more closely associated with prolonged bursts of electrical activity in affected muscle(s) rather than the short, irregular bursts of myoclonus. In addition, dystonic movements tend to have a sustained, directional nature rather than the random, flowing contractions seen with chorea. Dystonia also typically may be distinguished from the involuntary, rhythmic, “back-and-forth” movement characteristic of tremor. In some dystonic individuals, tremor-like muscle spasms or tremulous movements or dystonic tremor may be present upon attempting to actively resist abnormal, involuntary movements. Dystonias include, without limitation, a focal dystonia, a segmental dystonia, a multifocal dystonia, a generalized dystonia, and an acute dystonic reaction.

A focal dystonia refers to a dystonia where an individual has sustained involuntary muscle contractions limited to one area of the body. Focal dystonias often become apparent during the fourth or fifth decade, so called adult onset. However, symptoms may become obvious earlier in life. Overall, women are affected approximately three times more frequently than men. In up to 30% of individuals, focal dystonias may extend to involve nearby areas, resulting in segmental dystonia. Less commonly, symptoms may begin to affect certain non-adjacent regions (multifocal dystonia). Focal dystonia most typically affects those who rely on fine motor skills, such as, e.g., musicians, writers, and surgeons. It is generally “task specific,” meaning that it is only problematic during certain activities.

The symptoms associated with the focal dystonias are variable and depend upon the intensity and severity of the spasms and the specific body region and muscle groups involved. The rate of progression from symptom onset to difficulties in activities of daily living and disability are extremely variable, ranging from rapid development over days or weeks to a gradual progression over a decade or more. Symptoms of focal dystonias may initially be periodic, occurring only during stressful periods or random. At first, symptoms tend to appear when the affected body part performs certain movements; they typically disappear when the affected area is at rest. However, as the disease progresses, dystonic spasms begin to develop with other activities of the affected region. Symptoms may occur with voluntary actions involving other bodily areas. This phenomenon is known as overflow. Eventually, dystonia may be present when the affected part is at rest. Gradually, the affected area may assume an unusual and sometimes painful posture.

There are several forms of focal dystonia as well as other dystonias that may be limited to one area of the body. Focal dystonias include, without limitation, a cervical dystonia, a blepharospasm, a lingual dystonia, an oromandibular dystonia, a laryngeal dystonia, a limb dystonia, a truncal dystonia, an abdominal wall dystonia, and an anismus.

A cervical dystonia (also known as spasmodic torticollis) refers to sustained involuntary contractions of the neck (cervical) muscles and may be characterized by abnormal movements or postures of the neck and head. Dystonic spasms may result in jerky head movements or periodic or sustained unnatural position of the head. For example, the head may rotate to one side, to pull down towards the chest, or back, or a combination of these postures. There is also sideways or lateral rotation of the head and twisting or torticollis of the neck, often with head tilt. There may be isolated turning, flexing, or extending of the neck to the side (laterocollis), front (anterocollis), or back (retrocollis). One shoulder may be elevated and displaced forward on the side toward which the chin turns. In addition, there is often mild associated dystonia in the upper arm muscles on the same side (segmental dystonia). It is considered the most common form of focal dystonia.

Although cervical dystonia may become apparent at any age, symptoms usually begin between the ages 20 to 60 years. Women are affected approximately twice as commonly as men. Symptoms of cervical dystonia often worsen while walking or during stress. Symptoms typically improve with rest or sleep. Over two-thirds of individuals, particularly those with sustained head deviation, have associated neck pain. About one-third also experience head tremor (i.e., dystonic tremor), hand tremor, or both. Approximately 20% of individuals with cervical dystonia also have dystonic spasms of the eyelids (blepharospasm) or other muscles or of muscle groups of the arm or hand.

A blepharospasm (also known as dystonic blepharospasm) refers to sustained involuntary contractions of the muscles around the eyes. Dystonic spasms result in rapid blinking of the eyes or even intermittent or sustained forced closure of the eyelids causing effective blindness. Some individuals with blepharospasm experience relatively mild spasms of the muscle underlying the skin of the eyebrows and the root of the nose as well as of the middle and lower facial muscles. These spasms may result in grimacing or facial distortions.

In some individuals, blepharospasm may begin in just one eye (unilateral). Initial signs of the condition include eye irritation and burning, an increased sensitivity to light (photophobia), and excessive blinking. With disease progression, individuals may experience narrowing of the opening of the eyelids due to dystonia muscle contractions; involuntary, potentially forceful closure of the eyelids; and an inability to voluntarily raise the eyelids in order to open their eyes. Symptoms may worsen with stress, walking, reading, exposure to bright light, looking upward, watching television, or driving. Accordingly, blepharospasm may cause varying levels of difficulty with daily tasks, including reading and driving. Without treatment, blepharospasm often results in functional blindness, although vision may be normal. Blepharospasm affects women more frequently than men, with symptoms typically becoming apparent after age fifty.

In some individuals with blepharospasm, dystonic spasms may extend to nearby cranial areas, such as muscles of the tongue, mouth, jaw, neck, vocal cords, or other areas, thus becoming a segmental dystonia. The combination of blepharospasmodic contractions and oromandibular dystonia is called cranial dystonia or Meige's syndrome.

An oculogyric crisis refers to sustained involuntary contractions of the muscles from the eye and head. Dystonic spasms result in an extreme and sustained (usually) upward deviation of the eyes often with convergence causing diplopia. It is frequently associated with backwards and lateral flexion of the neck and either widely opened mouth or jaw clenching. Frequently a result of antiemetics such as, e.g., neuroleptics or metoclopramide, oculogyric crisis can also be caused by Chlorpromazine.

A lingual dystonia refers to sustained involuntary contractions of the muscles from the tongue. Dystonic spasms cause distortions of the tongue making eating and speaking difficult.

An oromandibular dystonia refers to sustained involuntary contractions of the muscles from the jaw and/or muscles from the tongue and may be characterized by distortions of the jaw, lower face, mouth and/or tongue. Involuntary contractions may involve the muscles used for chewing (masticatory muscles), as well as the thick muscle in the cheek that closes the jaw (buccinator muscles) and the broad muscle that draws back the lower jaw and closes the mouth (temporalis muscle). Some individuals may also experience involuntary contractions of the wide muscle at the side of the neck that close the jaws. This muscle draws down the corner of the mouth and lower lip (platysmal muscles) or other muscle groups. Dystonic spasms may extend to involve nearby areas including the muscles of the eyelids, nose, neck, or vocal cords. The combination of blepharospasm and oromandibular dystonia is called cranial dystonia or Meige's syndrome.

Associated findings of oromandibular dystonia may include spasms of jaw closure with difficulty opening the mouth (trismus) and clenching or grinding of the teeth (bruxism); spasms of jaw opening; or sideways deviation or protrusion of the jaw. Additional symptoms may also be present, such as lip tightening and pursing; drawing back (retraction) of the corners of the mouth; or deviation or protrusion of the tongue. Due to such findings, oromandibular dystonia may cause jaw pain as well as difficulties eating and speaking (dysarthria). In addition, in some individuals, the dystonic spasms may sometimes be provoked by certain activities, such as talking, chewing, or biting. As discussed earlier, particular activities or sensory tricks may sometimes temporarily alleviate oromandibular dystonia symptoms, including chewing gum, talking, placing a toothpick in the mouth, lightly touching the lips or chin, or applying pressure beneath the chin.

A laryngeal dystonia (also known as spasmodic dysphonia) refers to sustained involuntary contractions of the vocal cord muscles in the larynx and may be characterized by abnormal speech. Dystonic spasms may result in the voice to sound broken or reduces it to a whisper. This focal dystonia usually becomes apparent between ages 30 to 50 and affects women more frequently than men. Symptom onset is typically relatively gradual. Initial signs often include increased effort during speech and the loss of voice control that occurs with emotional stress. The condition tends to stabilize after about 1 to 2 years of increasing symptom severity. Speech may temporarily improve subsequent to sneezing or yawning. Laryngeal dystonia includes, e.g., adductor laryngeal dystonia and abductor laryngeal dystonia.

Adductor laryngeal dystonia involves the involuntary contraction of certain vocal muscles that draw the vocal cords together, causing the voice to have a restricted, strangled, or hoarse quality. Vocal expression is often interrupted by sudden, short pauses followed by abrupt bursts of speech, which may become less and less understandable. In most individuals, singing is not as severely affected as speech.

Abductor laryngeal dystonia involves the involuntary contraction of certain vocal muscles that draw the vocal cords apart causing the voice to have a breathy, whispering quality. Individuals suffering from this type of laryngeal dystonia tend to “run out of air” as they attempt to speak and are unable to speak loudly. As a result, their speech may also be difficult to understand.

A focal limb dystonia refers to sustained involuntary contractions of the muscles from an upper limb (arm; upper limb dystonia) or a lower limb (leg; lower limb dystonia). Dystonic spasms are usually accompanied by repetitive, twisting movements or abnormal positions or postures of the affected limb. The loss of precise muscle control and continuous unintentional movement results in painful cramping and abnormal positioning that makes continued use of the affected body parts impossible. Most focal limb dystonias are task-specific dystonias in that dystonic spasms typically occur in muscles or muscle groups only when performing activities requiring highly specialized, precise actions or extremely repetitive movements.

Upper limb dystonias typically affect a single muscle or small group of muscles in the wrist and/or hand and are generally known as focal hand dystonias. A focal hand dystonia is neurological in origin, and is not due to normal muscle fatigue. The most common type of focal hand dystonia is known as writer's cramp because it occurs when the individual is writing. Other types of focal hand dystonias have been reported among musicians, seamstresses, shoemakers, milkers, and participants in certain sports like golfers, tennis players, and dart throwers. Although most task-specific limb dystonias affect the upper limbs, they have been described in the lower limbs, such as among dancers, or cyclists.

A focal hand dystonia may often be characterized by an abnormally pronounced, forced grip on an object that typically occurs immediately upon grasping the object or shortly after using the object. Where grasping of an object is not performed, focal hand dystonia can cause involuntary curling of the fingers into the palm. Less commonly, there may be excessive extension of the fingers that causes the object to drop from the hand. Additional findings may include exaggerated flexion or extension of the affected wrist, forcing the palm of the hand downward or upward. Spasms may also extend to involve certain muscles of the arm and shoulder, potentially resulting in elevation of the elbow and outward extension of the shoulder. Performance of an activity with the object may be labored and shaky with discomfort or pain in the forearm. Touching or stabilizing the affected hand with the other hand may help to alleviate symptoms. In about 33% of individuals with a focal hand dystonia, dystonic spasms may eventually occur when other tasks are attempted or performed. Similarly, about 25% of individuals, dystonic spasms may extend to the previously unaffected hand.

Lower limb dystonias are a focal dystonia that primarily affect the ankle and foot, often resulting in inward turning of the heel with upward bending of the sole of the foot. The dystonic spasms initially occur only with walking (action dystonia). However, the dystonia may gradually be present at rest and eventually lead to sustained, fixed postures. Lower limb dystonia that appears during childhood is usually associated with the onset of generalized dystonia. However, lower limb dystonia that initially becomes evident during adulthood is rare. In such cases, diagnostic evaluations should be conducted to determine whether lower limb dystonia is present secondary to Parkinson's disease, parkinsonism syndromes, or other underlying causes.

A truncal dystonia refers to sustained involuntary contractions of the muscles from the back and torso. Dystonic spasms may cause unusual stretching, bending, or twisting of the trunk, sometimes accompanied with sideways curvature of the spine (scoliosis). At symptom onset, the spasms may occur only with standing or walking. Eventually, symptoms may also be present during rest. Dystonic spasms may eventually extend to involve adjacent regions, such as muscles of the upper arms or legs or the pelvis. This is a rare form of focal dystonia typically with an adult-onset appearance.

An abdominal wall dystonia (also known as belly-dancers dyskinesia) refers to sustained involuntary contractions of the muscles from the abdominal wall. Dystonic spasms may cause unusual writhing. This is a rare form of focal dystonia typically with an adult-onset appearance.

An anismus refers to a condition where sustained involuntary contractions of the muscles of the rectum. Dystonic spasms may result in painful defecation, constipation and may be complicated by encopresis.

A segmental dystonia refers to a dystonia where an individual has sustained muscle contractions affecting two or more nearby or contiguous areas of the body. This generally occurs when, after an onset of a focal dystonia, dystonic spasms spread to involve muscles or muscle groups from an additional area of the body adjacent to the initial focal dystonia. As many as 30% of individuals with a primary focal dystonia experience dystonic spasms in areas next to the primary site. Typically, an individual suffering from segmental dystonia has dystonic spasms involving facial and neck muscles; muscle groups of the neck and upper arm; or trunk and leg muscles. Cranial dystonia (Meige syndrome) is one common segmental dystonia that involves dystonic spasms of the muscles from the eyelids, jaw, mouth, and lower face. This condition is characterized by periodic or sustained closure of the eyelids (blepharospasm). Eyelid closure is accompanied by forceful spasms of jaw opening or closure, clenching or grinding of the teeth, sideways displacement of the jaw, lip tightening and pursing, and tongue protrusion. In addition, this form of segmental dystonia may spread to neck muscles or other muscle groups. Cranial dystonia more frequently affects women than men and typically becomes apparent during the sixth decade of life. Another common segmental dystonia is an oculogyric crisis.

A multifocal dystonia refers to a dystonia where an individual has sustained involuntary muscle contractions affecting two or more distant regions of the body. This generally occurs when, after an onset of a focal dystonia, dystonic spasms begin to affect involving muscles or muscle groups from a non-adjacent region or regions. For example, individuals affected with multifocal dystonia, may involve both legs; one or both arms and a leg; or the face and a leg.

A hemidystonia refers to a dystonia where an individual has sustained involuntary muscle contractions that affects one side of the body or is characterized by unilateral involvement of the upper and lower limbs. Hemidystonia typically occurs secondary to certain underlying conditions, particularly multiple sclerosis, tumor, stroke, or vascular malformations.

A generalized dystonia (also known as idiopathic torsion dystonia or dystonia musculrum deformans) refers to a dystonia where an individual has sustained involuntary muscle contractions throughout the body. Typically, an individual suffering from generalized dystonia has dystonic spasms involving muscles or muscle groups from both legs, or one leg and the back, as well as one other area of the body, such as, e.g., muscles or muscle group from one or both arms. The pattern of onset typically begins with leg involvement and then spreads upwards with eventual involvement of another region or regions of the body. Symptoms of a generalized dystonia usually manifest during childhood. Inheritable forms of a generalized dystonia are autosomal dominant.

An acute dystonic reaction refers to a dystonia brought about as an adverse response to certain types of medications. The most common medications include neuroleptics (antipsychotics), antiemetics, and antidepressants. An acute dystonic reaction can affect any part of the body including the arms and legs, trunk, neck, eyelids, face, or vocal cords. More men than women are affected and those between the age of 5-45 years are more often affected. Dystonic reactions are rarely seen in the elderly population. Alcohol and/or cocaine use increase the risk of developing a dystonic reaction.

A cerebral palsy refers to an involuntary movement disorder where an individual has difficulty controlling and coordinating muscles thereby affecting body movement, balance, and posture. An umbrella term for a group of disorders, cerebral palsy may involve muscle stiffness (spasticity), poor muscle tone, uncontrolled movements, and problems with posture, balance, coordination, walking, speech, swallowing, and many other functions. The severity of these problems varies widely, from very mild and subtle to very profound.

Cerebral palsy is caused by damage to the motor control centers of the developing brain and can occur during pregnancy, during childbirth or after birth up to about age three. Resulting limits in movement and posture cause activity limitation and are often accompanied by disturbances of sensation, depth perception and other sight-based perceptual problems, communication ability, and sometimes even cognition; sometimes a form of cerebral palsy may be accompanied by epilepsy. Cerebral palsy, no matter what the type, is often accompanied by secondary musculoskeletal problems that arise as a result of the underlying etiology. Cerebral palsy includes, without limitation, spastic palsy, dyskinetic palsy, and mixed palsy.

Spastic palsy (also known as hypertonic palsy or pyramidal palsy) refers to a condition where the muscles are stiff (spastic), and movements are jerky or awkward. Increased muscle tone is the defining characteristic of this type of palsy. Individuals with spastic palsy are hypertonic and have what is essentially a neuromuscular mobility impairment (rather than hypotonia or paralysis). Stemming from an upper motor neuron lesion in the brain as well as the corticospinal tract or the motor cortex, this damage impairs the ability of some nerve receptors in the spine to properly receive gamma amino butyric acid, leading to hypertonia in the muscles signaled by those damaged nerves. In any form of spastic palsy, clonus of the affected limb(s) may sometimes result, as well as muscle spasms resulting from the pain and/or stress of the tightness experienced. The spasticity can and usually does also lead to very early onset of muscle-stress symptoms like arthritis and tendinitis, especially in ambulatory individuals in their mid-20s and early-30s. Spastic cerebral palsy is the most common type of cerebral palsy, occurring in 70% to 80% of all cases.

Spastic palsy may be classified by which part of the body is affected, including, without limitation, a spastic monoplegia, a spastic diplegia, a spastic hemiplegia, a spastic triplegia, and a spastic quadriplegia. Spastic diplegia refers to a palsy condition that affects the lower limbs, with little to no upper-body spasticity. The most common form of spastic palsy (70-80% of known cases), most individuals with spastic diplegia are fully ambulatory, but are “tight” and have a scissors gait. Flexed knees and hips to varying degrees, and moderate to severe adduction (stemming from tight adductor muscles and comparatively weak abductor muscles), are present. Gait analysis is often done in early life on a semi-regular basis, and assistive devices are often provided like walkers, crutches or canes; any ankle-foot orthotics provided usually goes on both legs rather than just one. In addition, these individuals are often nearsighted. Over time, the effects of the spasticity sometimes produce hip problems and dislocations (see the main article and spasticity for more on spasticity effects). In three-quarters of spastic diplegics, also strabismus (crossed eyes) can be present as well.

Spastic hemiplegia refers to a palsy condition that affects one side of the body. Generally, injury to muscle-nerves controlled by the brain's left side will cause a right body deficit, and vice versa. Typically, individuals having spastic hemiplegia are ambulatory, although they generally have dynamic equinus (a limping instability) on the affected side and are primarily prescribed ankle-foot orthoses to prevent said equinus.

Spastic quadriplegia refers to a palsy condition that affects all four limbs more or less equally. Individuals with spastic quadriplegia are the least likely to be able to walk because their muscles are too tight and it is too much of an effort to do so. Some children with spastic quadriplegia also have hemiparetic tremors, an uncontrollable shaking that affects the limbs on one side of the body and impairs normal movement. Both spastic monoplegia, where only a single limb is affected, and spastic triplegia, where three limbs are affected, are also known forms of spastic palsy.

Dyskinetic palsy (also known as extrapyramidal palsy) refers to a condition affecting the coordination of movement. Dyskinetic palsy includes, without limitation, athetoid palsy and ataxic palsy. Athetoid palsy refers to a condition where the uncontrolled movements are slow and writhing. The movements can affect any part of the body, including the face, mouth, and tongue. Athetoid or dyskinetic cerebral palsy is mixed muscle tone—both hypertonia and hypotonia. Individuals with athetoid palsy have trouble holding themselves in an upright, steady position for sitting or walking, and often show involuntary motions. For some people with athetoid palsy, it takes a lot of work and concentration to get their hand to a certain spot (like scratching their nose or reaching for a cup). About 10-20% of cerebral palsy cases are of this type.

Ataxic palsy refers to a condition affecting balance and coordination. It is common for individuals to have difficulty with visual (e.g., depth perception) and/or auditory processing. If an individual can walk, the gait is most likely unsteady. In addition movements that are quick or require a great deal of control, such as, e.g., writing, typing, or using scissors may be difficult to perform. Individuals with ataxic palsy may also have hypotonia and tremors. About 5-10% of cases of cerebral palsy are of this type.

Hypotonic palsy refers to a condition where the musculature is limp, and an individual can move only a little or not at all.

Mixed palsy refers to a condition where there is a mixture of different types of cerebral palsy. One common combination is a spastic palsy with an athetoid palsy.

A periodic limb movement disorder (PLMD, or periodic movement of sleep), refers to an involuntary movement disorder where an individual exhibits involuntary movement of the limbs during sleep. The individual is often unaware of these movements. The etiology of PLMD is not known for certain, but individuals with PLMD may also suffer from other conditions such as narcolepsy or Parkinson's disease. Risk factors for PLMD are varied, and include caffeine, heart disease, sleep apnea, and doing physical activities near bedtime.

A restless leg syndrome (RLS, or Willis-Ekbom disease) refers to an involuntary movement disorder where an individual has an irresistible urge to move a part of one's body because of uncomfortable, odd, tickly or painful sensations. RLS is so named because the legs are usually the body part where the sensations are centered, but any part of the body could be affected, or even phantom limbs. The onset of RLS can occur at any age, and can be a progressive disease. The sensations generally begin or worsen while relaxing or trying to sleep, and most individuals experience their worst symptoms of RLS at night. Movement, such as walking or stretching, may bring relief of the sensations, but it is temporary—symptoms generally reappear when the movement ceases.

The symptoms of RLS, and their exacerbation with relaxation and nightfall, result in sleep disruption and difficulty in getting to sleep for many RLS sufferers. Additionally, a common side of effect of RLS is limb-jerking while the individual is asleep, which is another cause of sleep disruption. RLS-related sleep disruption in turn results in a significant impairment in the RLS sufferer's quality of life in general, due to the effects of drowsiness such as tardiness at work, impaired driving, poor mental acuity, etc. All of these daytime effects of RLS can translate into economic, safety and social concerns for the RLS sufferer and society.

RLS is classified as primary or secondary. Primary RLS is the idiopathic form, with no known cause. Primary RLS has a slow onset, and generally manifests at approximately 40-45 years of age. It tends to be progressive. Secondary RLS has a sudden onset, and generally manifests after age 40. It is generally associated with specific medical conditions or the use of certain drugs.

A spasm refers to an involuntary movement disorder where an individual has a sudden, involuntary contraction of muscles, hollow organ or orifice, which then ceases after a few minutes. A spasm may be accompanied by pain, and in severe cases can induce contractions so forceful as to tear tendons and ligaments. A disorder characterized by a series of spasms or permanent spasms is called a spasmism. Some causes of spasms include insufficient hydration, absence of electrolytes, and muscle overload. Spasms can also arise from a variety of medical conditions, including dystonias.

Hypertonic muscle spasms are spasms caused by residual tension in resting muscle (hypertonia), resulting from interruption of nerve feedback loops between the muscle and the brain. This interruption can be brought on, for example, by skeletal, circulatory, nerve, or inflammatory distress. Some hypertonic spasms, such as those caused by damage to the feedback nerves, is permanent unless treated.

Colic is a type of spasm which manifests as episodic pain due to the spasms of smooth muscle in an organ. The pain may induce nausea or vomiting in the individual if severe. Colic may bring on restlessness in an individual and the sensation of having to move about.

Geniospasm is a type of spasm which is a chronic involuntary movement disorder affecting the mentalis muscle. Geniospasm is a genetic disorder and manifests as rapid up and down movements of the chin and lower lip, beginning at childhood.

A stereotypy (also known as “punding” or “tweaking”, refers to an involuntary movement disorder where an individual displays repetitive and/or ritualistic movements. Stereotypies can be simple movements, for example rocking back and forth or flapping the arms, or more complex, for example walking in place or crossing and un-crossing the legs. Stereotypy usually begins at a very young age, with onset generally before the age of three, and can remain constant for years. Stereotypies are generally made worse by anxiety, fatigue, or stress. Stereotypy is associated with various disorders, including autism (where it is termed “stimming”), mental retardation, tardive dyskinesia, stereotypic movement disorder, and frontotemporal dementia. In the case of frontotemporal dementia, more than half of subjects exhibit stereotypies. It is also a side effect of some drug use. Stereotypy can be distinguished from a tic in that a stereotypy movement is generally a more ritualistic and patterned movement, less random than a tic movement, and involves more of the body or the entire body.

When stereotypy begins to cause impairment in an individual's normal functioning, the individual is often evaluated for stereotypic movement disorder (SMD). Some of the hallmarks of SMD are that the stereotypy movements generally begin in very early childhood and the SMD generally persists for four weeks or longer. Additionally, in SMD the stereotypy is not due to the effects of drug use or another condition, with the exception that SMD tends to affect children with developmental disorders. SMD can also result from head injury. The stereotypy of SMD manifests as repetitive involuntary movements that are of such severity that they can interfere with normal activities or even result in injury to the subject. Some examples of stereotypy in SMD include breath holding, gnashing of teeth (bruxism), banging the head, nail biting, nose picking, picking at the skin, thumb sucking, rocking back and forth, self-biting or hitting, and waving of the hands. There are currently no specific tests to diagnose SMD. SMD is often misdiagnosed as a tic; e.g., Tourette's. A tic, however, generally manifests around the age of six and comprises an involuntary movement that is unilateral and of short duration, whereas SMD generally manifests before age two and comprises an involuntary movement that is bilateral and of longer duration.

A tic refers to an involuntary movement disorder where an individual has a compulsive, repetitive movement or vocalization, the movement or vocalization being referred to as the tic (motor tic and phonic or vocal tic, respectively). Tic movements are generally unilateral and of short duration. Examples of tics include blinking and throat clearing. Tics generally manifest in childhood, at about age six. A subject may exhibit a tic as a result of a transient or chronic tic disorder, Tourette's disorder, or some other disorder (tic disorder NOS,” for “not otherwise specified”). With a transient tic disorder, a subject generally exhibits multiple motor and/or phonic tics, with the disorder lasting anywhere from four weeks to twelve months. With a chronic tic disorder, a subject generally exhibits motor or phonic tics, but not both types, with the disorder lasting over a year. With Tourette's disorder, a subject generally exhibits both motor and phonic tics, with the disorder lasting over a year. With a tic disorder NOS, a subject generally exhibits one or more tics that do not meet the criteria for any other specific tic disorder. Tic disorders are not due to the effects of medication or other medical conditions.

A tremor refers to an involuntary movement disorder wherein an individual has a rhythmic alteration of muscle contraction and relaxation involving oscillations or twitching of one or more body parts. Areas of the body that can be affected include the extremities, face, trunk, and vocal cords, with most tremors occurring in the hands. Tremor is most commonly classified by its clinical features and its cause. There are over twenty types of tremor generally recognized, and tremor is a symptom of various disorders including, for example, Wilson's disease. Some tremor classifications include, without limitation: cerebellar, dystonic, essential, orthostatic, Parkinsonian, physiologic, psychogenic, and rubral tremor.

Cerebellar tremor (or intention tremor) is a tremor of the extremities or head following a voluntary movement by the individual or while the individual maintains a particular posture. It is caused by damage to the cerebellum, which results in a tremor on the same side of the body as the damage. The cerebellar damage may result from stroke, tumor, multiple sclerosis, some other disease or inherited degenerative disorder, alcoholism, or overuse of certain medications. Cerebellar tremor may be accompanied by loss of muscle coordination (i.e., ataxia).

Dystonic tremor manifests in those affected by dystonia brought on by any of a variety of causes, and can be the initial sign of dystonia. This tremor affects any muscle and generally occurs when an individual makes particular movements.

Essential tremor (or benign essential tremor) is the most common type of tremor, and is not associated with any known pathology. Symptoms can appear at any age. It can be either nonprogressive or slowly progressive, in the latter case starting on one side of the body but gradually affecting both sides. The hands are most often affected but the head, voice, tongue, legs, and trunk may also be involved. Essential tremor may be accompanied by a mild disturbance in the gait disturbance. The severity of the tremors can increase with time, thus affecting the person's ability to perform certain tasks or activities of daily living. Some causes of essential tremor include emotions, stress, fever, exhaustion, and hypoglycemia.

Orthostatic tremor is characterized by fast, rhythmic muscle contractions in the legs and trunk immediately after standing. Tremor is accompanied by cramps in the legs.

Parkinsonian tremor is caused by damage to areas of the brain that control movement. It is often a precursor to Parkinson's disease, although this tremor can also be seen in other disorders. The tremor is classically seen in the hands as a “pill-rolling” action, but can also manifest in the chin, legs, trunk, or elsewhere. Parkinsonian tremor can be markedly increased by stress or emotions. The tremor generally starts on one side of the body but then usually progresses to include the other side.

Physiologic tremor occurs in normal individuals, generally not visible, and is not associated with any clinical pathologies. It can be heightened by a variety of factors, such as anxiety, exhaustion, hypoglycemia, stimulants, reaction to some drugs, or withdrawal from alcohol or other substances. It can manifest in any voluntary muscle group. “Enhanced physiologic tremor” is physiologic tremor that has advanced to more visible levels.

Psychogenic tremor (or hysterical tremor) occurs at rest or during movement. Characteristics of this tremor generally include sudden onset followed by remission, an increased incidence with stress, and changes in the body part affected. Many patients with psychogenic tremor have a psychiatric disease.

Rubral (or Holmes) tremor is characterized by a slow tremor that is present at rest or accompanying intended movements. It is associated with conditions affecting the midbrain, such as unusual strokes. Like cerebellar tremor, rubral tremor may also result from cerebellar damage.

Tremor can also be classified according to the position of the individual that most accentuates the tremor. These classifications include resting, contraction, posture and intention tremors. Resting tremors are worse when the individual is at rest. Examples of such tremors include Parkinsonian and severe essential tremors, and tremors induced by drug use such as antipsychotic drugs. Contraction tremors are worse when the muscles are contracted. Examples include essential and cerebellar tremors, and severe physiologic tremors as can arise from use of certain drugs. Posture tremors are worse when an aspect of the individual's posture is against gravity, as for example when the arms are raised. Examples include essential tremors and severe physiologic tremors. Intention tremors are worse when the individual makes an intentional (voluntary) movement, and can arise from disorders of the cerebellum.

Tremor can result from causes other than or in addition to those mentioned above. Some examples include alcoholism or alcohol withdrawal (resulting in a tremor known as asterixis), tobacco withdrawal, panic, cold, other neurological disorders, and damage to the nerves supplying the muscles.

A composition or compound is administered to an individual. An individual comprises all mammals including a human being. Typically, any individual who is a candidate for a conventional involuntary movement disorder treatment is a candidate for an involuntary movement disorder treatment disclosed herein. Pre-operative evaluation typically includes routine history and physical examination in addition to thorough informed consent disclosing all relevant risks and benefits of the procedure.

With reference to a therapy comprising a TEM, the amount of a TEM disclosed herein used with the methods of treatment disclosed herein will typically be an effective amount. As used herein, the term “effective amount” is synonymous with “therapeutically effective amount”, “effective dose”, or “therapeutically effective dose” and when used in reference to treating an involuntary movement disorder means the minimum dose of a TEM alone necessary to achieve the desired therapeutic effect and includes a dose sufficient to reduce a symptom associated with an involuntary movement disorder. An effective amount refers to the total amount of a TEM administered to an individual in one setting. As such, an effective amount of a TEM does not refer to the amount administered per site. The effectiveness of a TEM disclosed herein in treating an involuntary movement disorder can be determined by observing an improvement in an individual based upon one or more clinical symptoms, and/or physiological indicators associated with the condition. An improvement in an involuntary movement disorder also can be indicated by a reduced need for a concurrent therapy.

With reference to a standard dose combination therapy comprising a Clostridial toxin and a TEM, an effective amount of a Clostridial toxin is one where in combination with a TEM the amount of a Clostridial toxin achieves the desired therapeutic effect. For example, typically about 75-150 U of BOTOX® (Allergan, Inc., Irvine, Calif.), a BoNT/A, is administered in order to treat an involuntary movement disorder.

With reference to a low dose combination therapy comprising a Clostridial toxin and a TEM, an effective amount of a Clostridial toxin is one where in combination with a TEM the amount of a Clostridial toxin achieves the desired therapeutic effect, but such an amount administered on its own would be ineffective. For example, typically about 75-150 U of BOTOX® (Allergan, Inc., Irvine, Calif.), a BoNT/A, is administered in order to treat an involuntary movement disorder. However, in a low dose combination therapy, a suboptimal effective amount of BoNT/A would be administered to treat an involuntary movement disorder when such toxin is used in a combined therapy with a TEM. For example, less that 50 U, less than 25 U, less than 15 U, less than 10 U, less than 7.5 U, less than 5 U, less than 2.5 U, or less than 1 U of BoNT/A would be administered to treat an involuntary movement disorder when used in a low dose combination therapy with a TEM as disclosed herein.

The appropriate effective amount of a Clostridial toxin and/or a TEM to be administered to an individual for a particular sensory disturbance disorder can be determined by a person of ordinary skill in the art by taking into account factors, including, without limitation, the type of sensory disturbance disorder, the location of the involuntary movement disorder, the cause of the involuntary movement disorder, the severity of the involuntary movement disorder, the degree of relief desired, the duration of relief desired, the particular TEM and/or Clostridial toxin used, the rate of excretion of the particular TEM and/or Clostridial toxin used, the pharmacodynamics of the particular TEM and/or Clostridial toxin used, the nature of the other compounds to be included in the composition, the particular route of administration, the particular characteristics, history and risk factors of the individual, such as, e.g., age, weight, general health and the like, or any combination thereof. Additionally, where repeated administration of a composition disclosed herein is used, an effective amount of a Clostridial toxin and/or a TEM will further depend upon factors, including, without limitation, the frequency of administration, the half-life of the particular TEM and/or Clostridial toxin used, or any combination thereof. In is known by a person of ordinary skill in the art that an effective amount of a composition comprising a Clostridial toxin and/or TEM can be extrapolated from in vitro assays and in vivo administration studies using animal models prior to administration to humans.

Wide variations in the necessary effective amount are to be expected in view of the differing efficiencies of the various routes of administration. For instance, oral administration generally would be expected to require higher dosage levels than administration by intravenous or intravitreal injection. Similarly, systemic administration of a TEM would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a TEM disclosed herein that is administered can be adjusted accordingly.

In aspects of this embodiment, a therapeutically effective amount of a composition comprising a TEM reduces a symptom associated with an involuntary movement disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a composition comprising a TEM reduces a symptom associated with an involuntary movement disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a composition comprising a TEM reduces a symptom associated with an involuntary movement disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of the TEM is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a TEM generally is in the range of about 1 fg to about 3.0 mg. In aspects of this embodiment, an effective amount of a TEM can be, e.g., about 100 fg to about 3.0 mg, about 100 pg to about 3.0 mg, about 100 ng to about 3.0 mg, or about 100 μg to about 3.0 mg. In other aspects of this embodiment, an effective amount of a TEM can be, e.g., about 100 fg to about 750 μg, about 100 pg to about 750 μg, about 100 ng to about 750 μg, or about 1 μg to about 750 μg. In yet other aspects of this embodiment, a therapeutically effective amount of a TEM can be, e.g., at least 1 fg, at least 250 fg, at least 500 fg, at least 750 fg, at least 1 pg, at least 250 pg, at least 500 pg, at least 750 pg, at least 1 ng, at least 250 ng, at least 500 ng, at least 750 ng, at least 1 μg, at least 250 μg, at least 500 μg, at least 750 μg, or at least 1 mg. In still other aspects of this embodiment, a therapeutically effective amount of a composition comprising a TEM can be, e.g., at most 1 fg, at most 250 fg, at most 500 fg, at most 750 fg, at most 1 pg, at most 250 pg, at most 500 pg, at most 750 pg, at most 1 ng, at most 250 ng, at most 500 ng, at most 750 ng, at most 1 μg, at least 250 μg, at most 500 μg, at most 750 μg, or at most 1 mg.

In yet other aspects of this embodiment, a therapeutically effective amount of a TEM generally is in the range of about 0.00001 mg/kg to about 3.0 mg/kg. In aspects of this embodiment, an effective amount of a TEM can be, e.g., about 0.0001 mg/kg to about 0.001 mg/kg, about 0.03 mg/kg to about 3.0 mg/kg, about 0.1 mg/kg to about 3.0 mg/kg, or about 0.3 mg/kg to about 3.0 mg/kg. In yet other aspects of this embodiment, a therapeutically effective amount of a TEM can be, e.g., at least 0.00001 mg/kg, at least 0.0001 mg/kg, at least 0.001 mg/kg, at least 0.01 mg/kg, at least 0.1 mg/kg, or at least 1 mg/kg. In yet other aspects of this embodiment, a therapeutically effective amount of a TEM can be, e.g., at most 0.00001 mg/kg, at most 0.0001 mg/kg, at most 0.001 mg/kg, at most 0.01 mg/kg, at most 0.1 mg/kg, or at most 1 mg/kg.

In aspects of this embodiment, a therapeutically effective amount of a composition comprising a Clostridial toxin reduces a symptom associated with an involuntary movement disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a composition comprising a Clostridial toxin reduces a symptom associated with an involuntary movement disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a composition comprising a Clostridial toxin reduces a symptom associated with an involuntary movement disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a Clostridial toxin is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a Clostridial toxin generally is in the range of about 1 fg to about 30.0 μg. In other aspects of this embodiment, a therapeutically effective amount of a Clostridial toxin can be, e.g., at least 1.0 pg, at least 10 pg, at least 100 pg, at least 1.0 ng, at least 10 ng, at least 100 ng, at least 1.0 μg, at least 10 μg, at least 100 μg, or at least 1.0 mg. In still other aspects of this embodiment, a therapeutically effective amount of a Clostridial toxin can be, e.g., at most 1.0 pg, at most 10 pg, at most 100 pg, at most 1.0 ng, at most 10 ng, at most 100 ng, at most 1.0 μg, at most 10 μg, at most 100 pg, or at most 1.0 mg. In still other aspects of this embodiment, a therapeutically effective amount of a Clostridial toxin can be, e.g., about 1.0 pg to about 10 μg, about 10 pg to about 10 μg, about 100 pg to about 10 μg, about 1.0 ng to about 10 μg, about 10 ng to about 10 μg, or about 100 ng to about 10 μg. In still other aspects of this embodiment, a therapeutically effective amount of a Clostridial toxin can be from, e.g., about 1.0 pg to about 1.0 μg, about 10 pg to about 1.0 μg, about 100 pg to about 1.0 μg, about 1.0 ng to about 1.0 μg, about 10 ng to about 1.0 μg, or about 100 ng to about 1.0 μg. In other aspects of this embodiment, a therapeutically effective amount of a Clostridial toxin can be from, e.g., about 1.0 pg to about 100 ng, about 10 pg to about 100 ng, about 100 pg to about 100 ng, about 1.0 ng to about 100 ng, or about 10 ng to about 100 ng.

In yet other aspects of this embodiment, a therapeutically effective amount of a Clostridial toxin generally is in the range of about 0.1 U to about 2500 U. In other aspects of this embodiment, a therapeutically effective amount of a Clostridial toxin can be, e.g., at least 1.0 U, at least 10 U, at least 100 U, at least 250 U, at least 500 U, at least 750 U, at least 1,000 U, at least 1,500 U, at least 2,000 U, or at least 2,500 U. In still other aspects of this embodiment, a therapeutically effective amount of a Clostridial toxin can be, e.g., at most 1.0 U, at most 10 U, at most 100 U, at most 250 U, at most 500 U, at most 750 U, at most 1,000 U, at most 1,500 U, at most 2,000 U, or at most 2,500 U. In still other aspects of this embodiment, a therapeutically effective amount of a Clostridial toxin can be, e.g., about 1 U to about 2,000 U, about 10 U to about 2,000 U, about 50 U to about 2,000 U, about 100 U to about 2,000 U, about 500 U to about 2,000 U, about 1,000 U to about 2,000 U, about 1 U to about 1,000 U, about 10 U to about 1,000 U, about 50 U to about 1,000 U, about 100 U to about 1,000 U, about 500 U to about 1,000 U, about 1 U to about 500 U, about 10 U to about 500 U, about 50 U to about 500 U, about 100 U to about 500 U, about 1 U to about 100 U, about 10 U to about 100 U, about 50 U to about 100 U, about 0.1 U to about 1 U, about 0.1 U to about 5 U, about 0.1 U to about 10 U, about 0.1 U to about 15 U, about 0.1 U to about 20 U, about 0.1 U to about 25 U.

In still other aspects of this embodiment, a therapeutically effective amount of a Clostridial toxin generally is in the range of about 0.0001 U/kg to about 3,000 U/kg. In aspects of this embodiment, a therapeutically effective amount of a Clostridial toxin can be, e.g., at least 0.001 U/kg, at least 0.01 U/kg, at least 0.1 U/kg, at least 1.0 U/kg, at least 10 U/kg, at least 100 U/kg, or at least 1000 U/kg. In other aspects of this embodiment, a therapeutically effective amount of a Clostridial toxin can be, e.g., at most 0.001 U/kg, at most 0.01 U/kg, at most 0.1 U/kg, at most 1.0 U/kg, at most 10 U/kg, at most 100 U/kg, or at most 1000 U/kg. In yet other aspects of this embodiment, a therapeutically effective amount of a Clostridial toxin can be between, e.g., about 0.001 U/kg to about 1 U/kg, about 0.01 U/kg to about 1 U/kg, about 0.1 U/kg to about 1 U/kg, about 0.001 U/kg to about 10 U/kg, about 0.01 U/kg to about 10 U/kg, about 0.1 U/kg to about 10 U/kg about 1 U/kg to about 10 U/kg, about 0.001 U/kg to about 100 U/kg, about 0.01 U/kg to about 100 U/kg, about 0.1 U/kg to about 100 U/kg, about 1 U/kg to about 100 U/kg, or about 10 U/kg to about 100 U/kg. As used herein, the term “unit” or “U” is refers to the LD₅₀ dose, which is defined as the amount of a Clostridial toxin disclosed herein that killed 50% of the mice injected with the Clostridial toxin.

In aspects of this embodiment, a therapeutically effective amount of a standard or low combination therapy comprising a Clostridial toxin and a TEM reduces a symptom associated with an involuntary movement disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a standard or low combination therapy comprising a Clostridial toxin and a TEM reduces a symptom associated with an involuntary movement disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a standard or low combination therapy comprising a Clostridial toxin and a TEM reduces a symptom associated with an involuntary movement disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a standard or low combination therapy comprising a Clostridial toxin and a TEM is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a standard or low combination therapy comprising a Clostridial toxin and a TEM generally is in a Clostridial toxin: TEM molar ratio of about 1:1 to about 1:10,000. In other aspects of this embodiment, a therapeutically effective amount of a standard or low combination therapy comprising a Clostridial toxin and a TEM can be in a Clostridial toxin: TEM molar ratio of, e.g., about 1:1, about 1:2, about 1:5, about 1:10, about 1:25, about 1:50, about 1:75, about 1:100, about 1:200, about 1:300, about 1:400, about 1:500, about 1:600, about 1:700, about 1:800, about 1:900, about 1:1000, about 1:2000, about 1:3000, about 1:4000, about 1:5000, about 1:6000, about 1:7000, about 1:8000, about 1:9000, or about 1:10,000. In yet other aspects of this embodiment, a therapeutically effective amount of standard or low combination therapy comprising a Clostridial toxin and a TEM can be in a Clostridial toxin: TEM molar ratio of, e.g., at least 1:1, at least 1:2, at least 1:5, at least 1:10, at least 1:25, at least 1:50, at least 1:75, at least 1:100, at least 1:200, at least 1:300, at least 1:400, at least 1:500, at least 1:600, at least 1:700, at least 1:800, at least 1:900, at least 1:1000, at least 1:2000, at least 1:3000, at least 1:4000, at least 1:5000, at least 1:6000, at least 1:7000, at least 1:8000, at least 1:9000, or at least 1:10,000. In still other aspects of this embodiment, a therapeutically effective amount of a standard or low combination therapy comprising a Clostridial toxin and a TEM can be in a Clostridial toxin: TEM molar ratio of between, e.g., about 1:1 to about 1:10,000, about 1:10 to about 1:10,000, about 1:100 to about 1:10,000, about 1:500 to about 1:10,000, about 1:1000 to about 1:10,000, about 1:5000 to about 1:10,000, about 1:1 to about 1:1000, about 1:10 to about 1:1000, about 1:100 to about 1:1000, about 1:250 to about 1:1000, about 1:500 to about 1:1000, about 1:750 to about 1:1000, about 1:1 to about 1:500, about 1:10 to about 1:500, about 1:50 to about 1:500, about 1:100 to about 1:500, about 1:250 to about 1:500, about 1:1 to about 1:100, about 1:10 to about 1:100, about 1:25 to about 1:100, about 1:50 to about 1:100, or about 1:75 to about 1:100.

In yet other aspects of this embodiment, a therapeutically effective amount of a standard combination therapy comprising a Clostridial toxin and a TEM generally is in a range of about 0.50 U to about 250 U of Clostridial toxin and about 0.1 μg to about 2,000.0 μg of a TEM. In aspects of this embodiment, a therapeutically effective amount of a combined therapy comprising a Clostridial toxin and a TEM can be, e.g., about 0.1 U to about 10 U of a Clostridial toxin and about 10 μg to about 1,000 μg of a TEM, about 0.1 U to about 10 U of a Clostridial toxin and about 10 μg to about 500 μg of a TEM, about 0.1 U to about 10 U of a Clostridial toxin and about 10 μg to about 100 μg of a TEM, about 0.5 U to about 10 U of a Clostridial toxin and about 10 μg to about 1,000 μg of a TEM, about 0.5 U to about 10 U of a Clostridial toxin and about 10 μg to about 500 μg of a TEM, about 0.5 U to about 10 U of a Clostridial toxin and about 10 μg to about 100 μg of a TEM, about 1 U to about 10 U of a Clostridial toxin and about 100 μg to about 1,000 μg of a TEM, about 1 U to about 10 U of a Clostridial toxin and about 100 μg to about 500 μg of a TEM, or about 1 U to about 10 U of a Clostridial toxin and about 100 μg to about 100 μg of a TEM.

In yet other aspects of this embodiment, a therapeutically effective amount of a low combination therapy comprising a Clostridial toxin and a TEM generally is in a range of about 0.01 U to about 50 U of Clostridial toxin and about 0.1 μg to about 2,000.0 μg of a TEM. In aspects of this embodiment, a therapeutically effective amount of a combined therapy comprising a Clostridial toxin and a TEM can be, e.g., about 0.1 U to about 10 U of a Clostridial toxin and about 10 μg to about 1,000 μg of a TEM, about 0.1 U to about 10 U of a Clostridial toxin and about 10 μg to about 500 μg of a TEM, about 0.1 U to about 10 U of a Clostridial toxin and about 10 μg to about 100 μg of a TEM, about 0.5 U to about 10 U of a Clostridial toxin and about 10 μg to about 1,000 μg of a TEM, about 0.5 U to about 10 U of a Clostridial toxin and about 10 μg to about 500 μg of a TEM, about 0.5 U to about 10 U of a Clostridial toxin and about 10 μg to about 100 μg of a TEM, about 1 U to about 10 U of a Clostridial toxin and about 100 μg to about 1,000 μg of a TEM, about 1 U to about 10 U of a Clostridial toxin and about 100 μg to about 500 μg of a TEM, or about 1 U to about 10 U of a Clostridial toxin and about 100 μg to about 100 μg of a TEM.

Dosing can be single dosage or cumulative (serial dosing), and can be readily determined by one skilled in the art. For instance, treatment of an involuntary movement disorder may comprise a one-time administration of an effective dose of a composition disclosed herein. As a non-limiting example, an effective dose of a composition disclosed herein can be administered once to an individual, e.g., as a single injection or deposition at or near the site exhibiting a symptom of an involuntary movement disorder. Alternatively, treatment of an involuntary movement disorder may comprise multiple administrations of an effective dose of a composition disclosed herein carried out over a range of time periods, such as, e.g., daily, once every few days, weekly, monthly or yearly. As a non-limiting example, a composition disclosed herein can be administered once or twice yearly to an individual. The timing of administration can vary from individual to individual, depending upon such factors as the severity of an individual's symptoms. For example, an effective dose of a composition disclosed herein can be administered to an individual once a month for an indefinite period of time, or until the individual no longer requires therapy. A person of ordinary skill in the art will recognize that the condition of the individual can be monitored throughout the course of treatment and that the effective amount of a composition disclosed herein that is administered can be adjusted accordingly.

A composition disclosed herein can be administered to an individual using a variety of routes. Routes of administration suitable for a method of treating an involuntary movement disorder as disclosed herein include both local and systemic administration. Local administration results in significantly more delivery of a composition to a specific location as compared to the entire body of the individual, whereas, systemic administration results in delivery of a composition to essentially the entire body of the individual. Routes of administration suitable for a method of treating an involuntary movement disorder as disclosed herein also include both central and peripheral administration. Central administration results in delivery of a composition to essentially the central nervous system of an individual and includes, e.g., intrathecal administration, epidural administration as well as a cranial injection or implant. Peripheral administration results in delivery of a composition to essentially any area of an individual outside of the central nervous system and encompasses any route of administration other than direct administration to the spine or brain. The actual route of administration of a composition disclosed herein used can be determined by a person of ordinary skill in the art by taking into account factors, including, without limitation, the type of sensory disturbance disorder, the location of the involuntary movement disorder, the cause of the involuntary movement disorder, the severity of the involuntary movement disorder, the degree of relief desired, the duration of relief desired, the particular Clostridial toxin and/or TEM used, the rate of excretion of the Clostridial toxin and/or TEM used, the pharmacodynamics of the Clostridial toxin and/or TEM used, the nature of the other compounds to be included in the composition, the particular route of administration, the particular characteristics, history and risk factors of the individual, such as, e.g., age, weight, general health and the like, or any combination thereof.

In an embodiment, a composition disclosed herein is administered systemically to an individual. In another embodiment, a composition disclosed herein is administered locally to an individual. In an aspect of this embodiment, a composition disclosed herein is administered to a nerve of an individual. In another aspect of this embodiment, a composition disclosed herein is administered to the area surrounding a nerve of an individual.

A composition disclosed herein can be administered to an individual using a variety of delivery mechanisms. The actual delivery mechanism used to administer a composition disclosed herein to an individual can be determined by a person of ordinary skill in the art by taking into account factors, including, without limitation, the type of sensory disturbance disorder, the location of the involuntary movement disorder, the cause of the involuntary movement disorder, the severity of the involuntary movement disorder, the degree of relief desired, the duration of relief desired, the particular Clostridial toxin and/or TEM used, the rate of excretion of the Clostridial toxin and/or TEM used, the pharmacodynamics of the Clostridial toxin and/or TEM used, the nature of the other compounds to be included in the composition, the particular route of administration, the particular characteristics, history and risk factors of the individual, such as, e.g., age, weight, general health and the like, or any combination thereof.

In an embodiment, a composition disclosed herein is administered by injection. In aspects of this embodiment, administration of a composition disclosed herein is by, e.g., intramuscular injection, intraorgan injection, subdermal injection, dermal injection, intracranical injection, spinal injection, or injection into any other body area for the effective administration of a composition disclosed herein. In aspects of this embodiment, injection of a composition disclosed herein is to a nerve or into the area surrounding a nerve.

In another embodiment, a composition disclosed herein is administered by catheter. In aspects of this embodiment, administration of a composition disclosed herein is by, e.g., a catheter placed in an epidural space.

A composition disclosed herein as disclosed herein can also be administered to an individual in combination with other therapeutic compounds to increase the overall therapeutic effect of the treatment. The use of multiple compounds to treat an indication can increase the beneficial effects while reducing the presence of side effects.

Aspects of the present invention can also be described as follows:
1. A method of treating an involuntary movement disorder in an individual, the method comprising the step of administering to the individual in need thereof a therapeutically effective amount of a composition including a TEM, wherein administration of the composition reduces a symptom of the involuntary movement disorder, thereby treating the individual.
2. A use of a TEM in the manufacturing a medicament for treating an involuntary movement disorder in an individual in need thereof.
3. A use of a TEM in the treatment of an involuntary movement disorder in an individual in need thereof.
4. A method of treating an involuntary movement disorder in an individual, the method comprising the step of administering to the individual in need thereof a therapeutically effective amount of a composition including a Clostridial neurotoxin and a TEM, wherein administration of the composition reduces a symptom of the involuntary movement disorder, thereby treating the individual.
5. A use of a Clostridial neurotoxin and a TEM in the manufacturing a medicament for treating an involuntary movement disorder in an individual in need thereof.
6. A use of a Clostridial neurotoxin and a TEM in the treatment of an involuntary movement disorder in an individual in need thereof.
7. The embodiments of 1 to 6, wherein the TEM comprises a linear amino-to-carboxyl single polypeptide order of 1) a Clostridial toxin enzymatic domain, a Clostridial toxin translocation domain, a targeting domain, 2) a Clostridial toxin enzymatic domain, a targeting domain, a Clostridial toxin translocation domain, 3) a targeting domain, a Clostridial toxin translocation domain, and a Clostridial toxin enzymatic domain, 4) a targeting domain, a Clostridial toxin enzymatic domain, a Clostridial toxin translocation domain, 5) a Clostridial toxin translocation domain, a Clostridial toxin enzymatic domain and a targeting domain, or 6) a Clostridial toxin translocation domain, a targeting domain and a Clostridial toxin enzymatic domain.
8. The embodiments of 1 to 6, wherein the TEM comprises a linear amino-to-carboxyl single polypeptide order of 1) a Clostridial toxin enzymatic domain, an exogenous protease cleavage site, a Clostridial toxin translocation domain, a targeting domain, 2) a Clostridial toxin enzymatic domain, an exogenous protease cleavage site, a targeting domain, a Clostridial toxin translocation domain, 3) a targeting domain, a Clostridial toxin translocation domain, an exogenous protease cleavage site and a Clostridial toxin enzymatic domain, 4) a targeting domain, a Clostridial toxin enzymatic domain, an exogenous protease cleavage site, a Clostridial toxin translocation domain, 5) a Clostridial toxin translocation domain, an exogenous protease cleavage site, a Clostridial toxin enzymatic domain and a targeting domain, or 6) a Clostridial toxin translocation domain, an exogenous protease cleavage site, a targeting domain and a Clostridial toxin enzymatic domain.
9. The embodiments of 1 to 8, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
10. The embodiments of 1 to 9, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
11. The embodiments of 1 to 10, wherein the targeting domain is a sensory neuron targeting domain, a sympathetic neuron targeting domain, or a parasympathetic neuron targeting domain.
12. The embodiments of 1 to 10, wherein the targeting domain is an opioid peptide targeting domain, a galanin peptide targeting domain, a PAR peptide targeting domain, a somatostatin peptide targeting domain, a neurotensin peptide targeting domain, a SLURP peptide targeting domain, an angiotensin peptide targeting domain, a tachykinin peptide targeting domain, a Neuropeptide Y related peptide targeting domain, a kinin peptide targeting domain, a melanocortin peptide targeting domain, or a granin peptide targeting domain, a glucagon like hormone peptide targeting domain, a secretin peptide targeting domain, a pituitary adenylate cyclase activating peptide (PACAP) peptide targeting domain, a growth hormone-releasing hormone (GHRH) peptide targeting domain, a vasoactive intestinal peptide (VIP) peptide targeting domain, a gastric inhibitory peptide (GIP) peptide targeting domain, a calcitonin peptide targeting domain, a visceral gut peptide targeting domain, a neurotrophin peptide targeting domain, a head activator (HA) peptide, a glial cell line-derived neurotrophic factor (GDNF) family of ligands (GFL) peptide targeting domain, a RF-amide related peptide (RFRP) peptide targeting domain, a neurohormone peptide targeting domain, or a neuroregulatory cytokine peptide targeting domain, an interleukin (IL) targeting domain, vascular endothelial growth factor (VEGF) targeting domain, an insulin-like growth factor (IGF) targeting domain, an epidermal growth factor (EGF) targeting domain, a Transformation Growth Factor-β (TGβ) targeting domain, a Bone Morphogenetic Protein (BMP) targeting domain, a Growth and Differentiation Factor (GDF) targeting domain, an activin targeting domain, or a Fibroblast Growth Factor (FGF) targeting domain, or a Platelet-Derived Growth Factor (PDGF) targeting domain.
13. The embodiments of 8 to 12, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.
14. The embodiments of 1 to 13, wherein the Clostridial neurotoxin is a BoNT/A, a BoNT/B, a BoNT/C1, a BoNT/D, a BoNT/E, a BoNT/F, a BoNT/G, a TeNT, a BaNT, a BuNT, or any combination thereof.
15. The embodiments of 1 to 14, wherein the involuntary movement disorder is an ataxia, a chorea, a dyskinesia, a synkinesia, a dystonia, a cerebral palsy, a periodic limb movement disorder, a restless leg syndrome, a spasm, a stereotypy, a tic, or a tremor.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, compositions, methods or uses of treating an involuntary movement disorder.

### Example 1

### Treatment of an Ataxia

A female complains that she is experiencing gross lack of coordination of muscle movements that causes her to be unsteady when walking. After routine history and physical examination, a physician diagnosis the patient with an ataxia disorder involving abnormal sensory neuron activity and identifies the nerves and/or muscles involved in the condition. The woman is treated by injecting a composition comprising a TEM as disclosed in the present specification, targeting the nerves of the affected muscles. Alternatively, the woman may be treated by injecting a composition comprising a TEM and a suboptimal amount of a BoNT/A as disclosed in the present specification. The patient's condition is monitored and after about 2 weeks from treatment, the woman indicates that she can walk better and with a more normal gait. At one, three and five month check-ups, the woman indicates that she continues to walk in a normal manner and that her confidence in moving around has greatly increased. This increase in muscle coordination indicates a successful treatment with the composition comprising a TEM.

A similar treatment regime can be used to treat any chorea disorder including 1) an ataxia telangiectasia; 2) a Wilson's disease; 3) a cerebellar ataxia; 4) a sensory ataxia; or 5) a vestibular ataxia. Likewise, a similar therapeutic effect can be achieved with a suboptimal amount of any of the Clostridial toxins disclosed herein.

### Example 2

### Treatment of a Chorea

A man complains of rapid, involuntary movements that are not repetitive or rhythmic. After routine history and physical examination, a physician diagnosis the patient with a chorea disorder involving abnormal sensory neuron activity and identifies the nerves and/or muscles involved in the condition. The man is treated by injecting a composition comprising a TEM as disclosed in the present specification, targeting the nerves of the affected muscles. Alternatively, the man may be treated by injecting a composition comprising a TEM and a suboptimal amount of a BoNT/A as disclosed in the present specification. The patient's condition is monitored and after about 2 weeks from treatment, the man indicates that he is not experiencing the rapid, involuntary movements as much as before the treatment. At one and three month check-ups, the man indicates that the involuntary movements have greatly subsided. This decrease in rapid, involuntary movements indicates a successful treatment with the composition comprising a TEM.

A similar treatment regime can be used to treat any ataxia disorder including 1) a Sydenham's chorea; 2) a chorea gravidarum; 3) a Wilson's disease; 4) a McLeod syndrome; 5) a hemiballismus; or 6) a ballism. Likewise, a similar therapeutic effect can be achieved with a suboptimal amount of any of the Clostridial toxins disclosed herein.

### Example 3

### Treatment of a Dyskinesia

A man complains of involuntary body movements, but diminished voluntary movements, a condition that developed when the patient began taking schizophrenia medication. After routine history and physical examination, a physician diagnosis the patient with a tardive dyskinesia involving abnormal sensory neuron activity and identifies the nerves and/or muscles involved in the condition. The man is treated by intradermally injecting a composition comprising a TEM as disclosed in the present specification, targeting the paraspinal muscles. Alternatively, the man may be treated by injecting a composition comprising a TEM and a suboptimal amount of a BoNT/A as disclosed in the present specification. The patient's condition is monitored and after about 2 weeks from treatment, the man indicates that he is not experiencing the involuntary movements as much as before the treatment and has regained normal voluntary movements. At one and six month check-ups, the man indicates that the involuntary movements have greatly subsided and his voluntary movements are normal. This decrease in involuntary movements and experiencing normal voluntary movements indicates a successful treatment with the composition comprising a TEM.

A similar treatment regime can be used to treat any other dyskinesia disorder including 1) a tardive akathisia; 2) a tardive dystonia; 3) a tardive myoclonus; and 4) a tardive tourettism. Likewise, a similar therapeutic effect can be achieved with a suboptimal amount of any of the Clostridial toxins disclosed herein.

### Example 4

### Treatment of a Synkinesia

A female complains that she is experiencing involuntary squinting when she voluntarily smiles. After routine history and physical examination, a physician diagnosis the patient with a synkinesia disorder involving abnormal sensory neuron activity and identifies the nerves and/or muscles involved in the condition. The woman is treated by injecting a composition comprising a TEM as disclosed in the present specification, targeting the nerves of the affected muscles. Alternatively, the woman may be treated by injecting a composition comprising a TEM and a suboptimal amount of a BoNT/A as disclosed in the present specification. The patient's condition is monitored and after about 2 weeks from treatment, the woman indicates that she no longing squints her eyes when she smiles. At one, three and five month check-ups, the woman indicates that she continues to not squint when she smiles. This decrease in the involuntary squinting when she voluntarily smiles indicates a successful treatment with the composition comprising a TEM.

A similar therapeutic effect can be achieved with a suboptimal amount of any of the Clostridial toxins disclosed herein.

### Example 5

### Treatment of a Dystonia

A woman complains of muscle contractions that twist her head in several directions, including her chin being pulled toward either shoulder, her chin being pulled up, and her chin being pulled down. The woman also complains of jerking motions of her head, as well as occasional shoulder elevations and arm tremors. She has failed to respond to numerous medications including standard botulinum toxin treatments, like BoNT/A and BoNT/B. After routine history and physical examination, a physician identifies the muscles involved in the abnormal postures and movements and orders an electromyogram (EMG) to test nerve function. Based on these examinations, the physician diagnosis the patient with a cervical dystonia and identifies the nerves and/or muscles involved in the condition. The woman is treated by injecting at multiple points along the muscles a composition comprising a TEM and a suboptimal amount of a BoNT/A as disclosed in the present specification. The patient's condition is monitored and after about 2 days from treatment, the woman indicates she has decreased tremors and muscle contractions. At two and four month check-ups, the woman indicates decrease in tremors and muscle contractions continue, and as a result the pain has subsided. This decrease in decrease in tremors and muscle contractions indicates a successful treatment with the composition comprising a TEM and a BoNT/A as disclosed in the present specification.

A similar treatment regime can be used to treat any dystonia including 1) a focal dystonia like a cervical dystonia, a blepharospasm, a lingual dystonia, an oromandibular dystonia, a laryngeal dystonia, a limb dystonia, a truncal dystonia, an abdominal wall dystonia, and an anismus; 2) a segmental dystonia like an oculogyric crisis or a cranial dystonia; 3) a multifocal dystonia; 4) a generalized dystonia; or 6) an acute dystonic reaction. Likewise, a similar therapeutic effect can be achieved with a suboptimal amount of any of the Clostridial toxins disclosed herein.

### Example 6

### Treatment of a Cerebral Palsy

An male with cerebral palsy since birth complains about the difficulty controlling and coordinating muscles thereby affecting body movement, balance, and posture. Unfortunately, the patient has weakness in his arms that precludes standard botulinum toxin treatment. Instead, his physician treats the man by injecting at multiple points along the affected muscles a composition comprising a TEM and a suboptimal amount of a BoNT/A as disclosed in the present specification. The patient's condition is monitored and after about 2 days from treatment, the man indicates he can better control and coordinate his muscle movements At two and four month check-ups, the man indicates he has had continued control and coordination in his muscle movements. This increased control and coordination in muscle movements indicates a successful treatment with the composition comprising a TEM and a BoNT/A as disclosed in the present specification.

A similar treatment regime can be used to treat any palsy including a spastic palsy, a dyskinetic palsy, a hypotonic palsy, or a mixed palsy. Likewise, a similar therapeutic effect can be achieved with a suboptimal amount of any of the Clostridial toxins disclosed herein.

### Example 7

### Treatment of a Periodic Limb Movement Disorder

A man complains about experiencing involuntary limb movements while he is asleep that either awaits him or disturb his wife, awaking her. After routine history and physical examination, a physician diagnosis the patient with a periodic limb movement disorder involving abnormal sensory neuron activity and identifies the nerves and/or muscles involved in the condition. The man is treated by injection of a composition comprising a TEM as disclosed in the present specification, targeting the nerves of the affected muscles. Alternatively, the man may be treated by injecting a composition comprising a TEM and a suboptimal amount of a BoNT/A as disclosed in the present specification. The patient's condition is monitored and after about 2 weeks from treatment, the man indicates he has not experienced involuntary muscle movements while sleeping. At two and four month check-ups, the man indicates that he continues to sleep through the night without experiencing involuntary movements. This decrease in involuntary movements while asleep indicates a successful treatment with the composition comprising a TEM.

A similar therapeutic effect can be achieved with a suboptimal amount of any of the Clostridial toxins disclosed herein.

### Example 8

### Treatment of a Restless Leg Syndrome

A man complains about experiencing aches in both calves, accompanied by an irresistible impulse to move his legs. After routine history and physical examination, a physician diagnosis the patient with a restless leg syndrome involving abnormal sensory neuron activity and identifies the nerves and/or muscles involved in the condition. The man is treated by injection of a composition comprising a TEM as disclosed in the present specification, targeting the nerves of the affected limbs. Alternatively, the man may be treated by injecting a composition comprising a TEM and a suboptimal amount of a BoNT/A as disclosed in the present specification. The patient's condition is monitored and after about 2 weeks from treatment, the man indicates he has not experienced irresistible impulse to move his legs. At three and six month check-ups, the man indicates that he continues to not experience irresistible impulse to move his legs and the aches in his calves has gone. This decreased impulse to move his legs indicates a successful treatment with the composition comprising a TEM.

A similar treatment regime can be used to treat either a primary or secondary restless leg syndrome regardless of the body part affected or even for a phantom limb situation. Likewise, a similar therapeutic effect can be achieved with a suboptimal amount of any of the Clostridial toxins disclosed herein.

### Example 9

### Treatment of a Spasm

A man complains about experiencing sudden, involuntary muscle contraction which then ceases after a few minutes. After routine history and physical examination, a physician diagnosis the patient with a spasm disorder involving abnormal sensory neuron activity and identifies the nerves and/or muscles involved in the condition. The man is treated by injection of a composition comprising a TEM as disclosed in the present specification, targeting the nerves of the affected limbs. Alternatively, the man may be treated by injecting a composition comprising a TEM and a suboptimal amount of a BoNT/A as disclosed in the present specification. The patient's condition is monitored and after about 2 weeks from treatment, the man indicates he has not experienced a spasm. At three and six month check-ups, the man indicates that he continues to not experience any spasms and is leading a satisfying life. This decrease in spasms indicates a successful treatment with the composition comprising a TEM.

A similar treatment regime can be used to treat any spasm including 1) a hypertonic muscle spasm; 2) a colic spasm; or 3) a geniospasm. Likewise, a similar therapeutic effect can be achieved with a suboptimal amount of any of the Clostridial toxins disclosed herein.

### Example 10

### Treatment of a Stereotypy

A woman complains about experiencing involuntary repetitive or ritualistic movements like rocking back and forth. After routine history and physical examination, a physician diagnosis the patient with a stereotypy disorder involving abnormal sensory neuron activity and identifies the nerves and/or muscles involved in the condition. The woman is treated by injection of a composition comprising a TEM as disclosed in the present specification, targeting the nerves of the affected muscles. Alternatively, the woman may be treated by injecting a composition comprising a TEM and a suboptimal amount of a BoNT/A as disclosed in the present specification. The patient's condition is monitored and after about 2 weeks from treatment, the woman indicates she has noticed a reduction in these involuntary movements. At three and six month check-ups, the woman indicates that she continues to not experience any involuntary repetitive or ritualistic movements. This decrease in these involuntary movements indicates a successful treatment with the composition comprising a TEM.

A similar treatment regime can be used to treat any stereotypy disorder including stereotypic movement disorder. Likewise, a similar therapeutic effect can be achieved with a suboptimal amount of any of the Clostridial toxins disclosed herein.

### Example 11

### Treatment of a Tic

A man complains about experiencing a compulsive, repetitive blinking which then ceases after a few minutes. After routine history and physical examination, a physician diagnosis the patient with a tic disorder involving abnormal sensory neuron activity and identifies the nerves and/or muscles involved in the condition. The man is treated by injection of a composition comprising a TEM as disclosed in the present specification, targeting the nerves of the affected limbs. Alternatively, the man may be treated by injecting a composition comprising a TEM and a suboptimal amount of a BoNT/A as disclosed in the present specification. The patient's condition is monitored and after about 2 weeks from treatment, the man indicates he has not experienced a tic. At three and six month check-ups, the man indicates that he continues to not experience any tics and is leading a satisfying life. This decrease in compulsive, repetitive blinking indicates a successful treatment with the composition comprising a TEM.

A similar treatment regime can be used to treat any tic disorder including 1) a motor tic; 2) a phonic tic; or 3) a vocal tic. Likewise, a similar therapeutic effect can be achieved with a suboptimal amount of any of the Clostridial toxins disclosed herein.

### Example 12

### Treatment of a Tremor

A woman complains about experiencing involuntary rhythmic twitching of her hands and lower arms. After routine history and physical examination, a physician diagnosis the patient with a tremor disorder involving abnormal sensory neuron activity and identifies the nerves and/or muscles involved in the condition. The woman is treated by injection of a composition comprising a TEM as disclosed in the present specification, targeting the nerves of the affected muscles. Alternatively, the woman may be treated by injecting a composition comprising a TEM and a suboptimal amount of a BoNT/A as disclosed in the present specification. The patient's condition is monitored and after about 2 weeks from treatment, the woman indicates she has noticed a reduction in these tremors. At three and six month check-ups, the woman indicates that she continues to not experience any tremors in her arm or hands. This decrease in tremors indicates a successful treatment with the composition comprising a TEM.

A similar treatment regime can be used to treat any stereotypy disorder including stereotypic movement disorder. Likewise, a similar therapeutic effect can be achieved with a suboptimal amount of any of the Clostridial toxins disclosed herein.

### Example 13

### Treatment of Cervical Dystonia Involving Retrocollis

A man with a history of cervical dystonia involving retrocollis, associated with a prominent sensory trick and neck pain. He has been treated with a range of Botox® dosing starting at 250 units and ranging up to 500 units over multiple treatment cycles. His pain improves but neck weakness occurs as the dose is titrated upwards and this limits additional benefit for his neck position. As a result his dose of Botox is reduced to 50 units in cervical paraspinal muscles only. No deep injections are given. No neck weakness occurs. This sub-therapeutic dose results in pain relief and improved head posture without weakness.

Patient could also be treated with a therapeutically effect amount of a composition comprising a TEM as disclosed in the present specification.

### CONCLUSION

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating an involuntary movement disorder in an individual, the method comprising the step of administering to the individual in need thereof a therapeutically effective amount of a composition including a TEM comprising a targeting domain, a Clostridial toxin translocation domain and a Clostridial toxin enzymatic domain, wherein the targeting domain is a sensory neuron targeting domain, a sympathetic neuron targeting domain, or a parasympathetic neuron targeting domain, and wherein administration of the composition reduces a symptom of the involuntary movement disorder, thereby treating the individual.

2. The method of claim 1, wherein the TEM comprises a linear amino-to-carboxyl single polypeptide order of 1) the Clostridial toxin enzymatic domain, the Clostridial toxin translocation domain, the targeting domain, 2) the Clostridial toxin enzymatic domain, the targeting domain, the Clostridial toxin translocation domain, 3) the targeting domain, the Clostridial toxin translocation domain, and the Clostridial toxin enzymatic domain, 4) the targeting domain, the Clostridial toxin enzymatic domain, the Clostridial toxin translocation domain, 5) the Clostridial toxin translocation domain, the Clostridial toxin enzymatic domain and the targeting domain, or 6) the Clostridial toxin translocation domain, the targeting domain and the Clostridial toxin enzymatic domain.

3. The method of claim 1, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.

4. The method of claim 1, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.

5. The method of claim 1, wherein the involuntary movement disorder is an ataxia, a chorea, a dyskinesia, a synkinesia, a dystonia, a cerebral palsy, a periodic limb movement disorder, a restless leg syndrome, a spasm, a stereotypy, a tic, or a tremor.

6. A method of treating an involuntary movement disorder in an individual, the method comprising the step of administering to the individual in need thereof a therapeutically effective amount of a composition including a TEM comprising a targeting domain, a Clostridial toxin translocation domain, a Clostridial toxin enzymatic domain, and an exogenous protease cleavage site, wherein the targeting domain is a sensory neuron targeting domain, a sympathetic neuron targeting domain, or a parasympathetic neuron targeting domain, and wherein administration of the composition reduces a symptom of the involuntary movement disorder, thereby treating the individual.

7. The method of claim 6, wherein the TEM comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.

8. The method of claim 6, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.

9. The method of claim 6, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.

10. The method of claim 6, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.

11. The method of claim 6, wherein the involuntary movement disorder is an ataxia, a chorea, a dyskinesia, a synkinesia, a dystonia, a cerebral palsy, a periodic limb movement disorder, a restless leg syndrome, a spasm, a stereotypy, a tic, or a tremor.

12. A use of a TEM in the manufacturing a medicament for treating an involuntary movement disorder in an individual in need thereof, wherein the TEM comprising a targeting domain, a Clostridial toxin translocation domain and a Clostridial toxin enzymatic domain, wherein the targeting domain is a sensory neuron targeting domain, a sympathetic neuron targeting domain, or a parasympathetic neuron targeting domain.

13. A use of a TEM in the manufacturing a medicament for treating an involuntary movement disorder in an individual in need thereof, wherein the TEM comprising a targeting domain, a Clostridial toxin translocation domain, a Clostridial toxin enzymatic domain, and an exogenous protease cleavage site, wherein the targeting domain is a sensory neuron targeting domain, a sympathetic neuron targeting domain, or a parasympathetic neuron targeting domain.

14. A use of a TEM in the treatment of an involuntary movement disorder in an individual in need thereof, wherein the TEM comprising a targeting domain, a Clostridial toxin translocation domain and a Clostridial toxin enzymatic domain, wherein the targeting domain is a sensory neuron targeting domain, a sympathetic neuron targeting domain, or a parasympathetic neuron targeting domain.

15. A use of a TEM in the treatment of an involuntary movement disorder in an individual in need thereof, the use comprising the step of administering to the individual a therapeutically effective amount of the composition, wherein the TEM comprising a targeting domain, a Clostridial toxin translocation domain, a Clostridial toxin enzymatic domain, and an exogenous protease cleavage site, wherein the targeting domain is a sensory neuron targeting domain, a sympathetic neuron targeting domain, or a parasympathetic neuron targeting domain.
